(19) United States
(12) Patent Application Publication
(10) Pub. No.: US 2026/0294308 A1
(43) Pub. Date: Oct. 1, 2026

(54) AN ELECTRODE WITH ULTRALOW BIOELECTRONIC IMPEDANCE BASED ON MOLECULE ANCHORING

(71) Applicant: Nanyang Technological University, Singapore (SG)

(72) Inventors: Liang PAN, Singapore (SG); Xiaodong CHEN, Singapore (SG)

(21) Appl. No.: 18/880,356

(22) PCT Filed: Jul. 28, 2023
(86) PCT No.: PCT/SG2023/050527

§ 371 Date: Dec. 31, 2024

(30) Foreign Application Priority Data
Jul. 28, 2022 (SG) 10202250616F

(51) Int. Cl. A61B 5/266; A61B 5/259; A61B 5/296; A61F 2/72; C09J 9/02; C09J 139/04

(52) U.S. Cl. CPC A61B 5/266; A61B 5/259; A61B 5/296; A61F 2/72; C09J 9/02; C09J 139/04; A61B 2562/164

(57) ABSTRACT

Disclosed herein is an ionic conducting gel that comprises a polymeric adhesive material in an amount of from 5 to 20 wt % of the gel, a metal salt in the amount of from 2 to 10 wt % of the gel, and the balance being a hydrophilic polymeric material, wherein a gel matrix is formed by intermolecular interactions between the polymeric adhesive material and the hydrophilic polymeric material, and the metal salt is distributed throughout the gel matrix. Also disclosed herein are a flexible electrode device, comprising a flexible substrate material having a first surface and a second surface, a metal electrode pad on the first surface of the substrate, and an ionic conducting gel layer that covers the metal electrode pad, and a prosthetic control system comprising one or more electrode devices.

### FIELD OF INVENTION

The current invention generally relates to ionic conducting gels, and more particularly relates to ionic conducting gels comprising a polymeric adhesive material and a metal salt, and are suitable for use in flexible electrode devices and prosthetic control systems.

### BACKGROUND

The listing or discussion of a prior-published document in this specification should not necessarily be taken as an acknowledgement that the document is part of the state of the art or is common general knowledge.

Myoelectric control is an advanced technique for amputees concerned with detecting, recognizing and applying the lossless myoelectric signals to control prostheses. However, Due to long-term inactivity-caused fibrosis and atrophy of an amputee's skin, the poor ionic-electronic coupling efficiency at the skin-electrode interface can easily distort the low-level myoelectric signals, especially from <10% maximal voluntary contraction (MVC).

Disliking but relying on their electric prostheses is the typical feeling of amputees, but this dislike causes the eventual mean rejection and abandonment rate for these prostheses to reach 35% (Biddiss, E. A. & Chau, T. T. Prosthet. Orthot. Int. 2007, 31, 236). To realize the amputees' ultimate dream: human and prosthesis in one, attempts to design a myoelectric prosthesis with a capability and comfort that is comparable to human limbs has been actively explored (Craelius, W. Science, 2002, 295, 1018; Hochberg, L. R. et al., Nature, 2012, 485, 372). As a fundamental premise, a high-fidelity myoelectric signal without distortion can ensure the accurate identification of phantom limb activities for precise prosthetic control. Myoelectric signals are electrical action potentials generated during muscle contraction. These signals contain neural information sent from the brain to control a specific movement, making them usable as input sources for prosthesis control by amputees who are trained to generate them (Kaur, M. et al., Adv. Mater., 2021, 33, 2002534; Craelius, W., Science, 2002, 295, 1018; Hochberg, L. R. et al., Nature, 2012, 485, 372). Recording myoelectric signals is made possible through non-invasive electrodes placed on the skin of the stump. However, these signals are highly variable and are usually normalized to the maximum voluntary contraction (MVC) of the muscle. For fine and dexterous movements such as finger and wrist movements, the muscle contraction is below 10% MVC (Alenabi, T. et al., J. Shoulder Elbow Surg., 2013, 22, 1400; Martin, B. J. et al., Hum. Factors, 1996, 38, 654). The challenge is that the myoelectric signals generated from these movements are frequently distorted and have low signal-to-noise ratios (SNRs), as traditional electrodes do not effectively couple with the amputee's skin. As a result, amputees often forced to use stronger muscle contraction (above 10% MVC) in order to trigger the recognition of signals for weak but fine finger movements. This mismatch between the amputee's muscle contraction and prosthetic movement produces a physiological conflict between the new reflex arc and quondam muscle memory. It also generates substantial discordance and excessive friction at the prosthesis-stump interface, resulting in discomfort, excessive sweating, skin irritation, and the eventual abandonment of myoelectric prosthesis (Biddiss, E. A. & Chau, T. T., Prosthet. Orthot. Int., 2007, 31, 236; Gajewski, D. & Granville, R., J. Am. Acad. Orthop. Surg., 2006, 14, S183). To address this mismatch and give amputees more precise control over their prosthesis, improving the quality and availability of the low-level myoelectrical signals (especially those less than 10% MVC) is critical to compensate for that mismatching.

The detection of myoelectric signals involves capacitive coupling of ionic fluxes in the skin and electronic currents in gel electrodes through two electric double layers (EDLs) at the skin-electrode interface (FIG. 1) (Yang, C. & Suo, Z., Nat. Rev. Mater., 2018, 3, 125; Yuk, H. et al., Chem. Soc. Rev., 2019, 48, 1642; Cai, P. et al., Nat. Commun., 2020, 11, 2183). Traditionally, methods to enhance ionic-electronic coupling and reduce bioelectronic impedance have focused on making electrodes more flexible and stretchable (King, D. R. et al., Adv. Mater., 2014, 26, 4345; Miyamoto, A. et al., Nat. Nanotechnol., 2017, 12, 907; Pan, S. et al., Adv. Funct. Mater., 2020, 30, 1909540) or improving the interaction between the electrodes and the skin's surface molecules (Pan, L. et al., Adv. Mater., 2020, 32, 2003723; Luo, Y. et al., Adv. Mater., 2021, 33, 2007848) to conform to the curved skin. However, these approaches have only improved the detection of low-level signals in normal skin. Capacitive coupling remains weak on amputated skin because of changes in the inner skin such as fibrosis and atrophy caused by amputation. This results in imbalanced intra- and extra-cellular calcium concentration in amputated skin due to prolonged inactivity (Lannan, F. M. & Meyerle, J. H., Cutis, 2019, 103, 86), which decreases the induced charge at the electrode metal layer during capacitive coupling (Parekh, A. B., J. Physciol., 2003, 547, 333; Agrawal, A. et al., J. Cell Commun. Signal., 2018, 12, 645). This imbalance elevates the level of interfacial impedance from values close to that would more closely mimic the intrinsic impedance of humans (6-10 kΩ) to a range of from high kΩ to low MΩ, making it impossible to record myoelectric signals below 10% MVC with high quality (which is critical to compensate for this mismatching). It is noted that approximately 80% of the bioelectric impedance (the so-called barrier effect) arises from the outermost layer of the skin, the stratum corneum (SC) (Wu, K. S. et al., Biomaterials, 2006, 27, 785; and Kabiri Ameri, S. et al., ACS Nano, 2017, 11, 7634).

### SUMMARY OF INVENTION

It has been surprisingly found that the generation and use of an ionic conducting gel in the production of gel electrodes that firmly anchor into the skin reduce the barrier effect noted above and increase the ionic-electronic coupling regions. Thus, the current invention relates to said ionic conducting gel, its use in a gel electrode and use of said electrode in a prosthetic device.

Aspects and embodiments of the invention will now be discussed by reference to the following numbered clauses.

1. An ionic conducting gel that comprises:
a polymeric adhesive material in an amount of from 5 to 20 wt % of the gel;
a metal salt in the amount of from 2 to 10 wt % of the gel; and
the balance being a hydrophilic polymeric material, wherein a gel matrix is formed by between the polymeric adhesive material and the hydrophilic polymeric material, and the metal salt is distributed throughout the gel matrix.

2. The ionic conducting gel according to Clause 1, wherein the number average molecular weight of the hydrophilic polymeric material is from 200 to 15,000 Daltons.

3. The ionic conducting gel according to Clause 1 or Clause 2, wherein the hydrophilic polymeric material is a polyethylene glycol or a polyalcohol.

4. The ionic conducting gel according to Clause 3, wherein the hydrophilic polymeric material is a polyethylene glycol, optionally wherein the polyethylene glycol has a number 30 average molecular weight of 200.

5. The ionic conducting gel according to any one of the preceding clauses, wherein the polymeric adhesive material is selected from one or more of the group consisting of a polysaccharide, a cellulose hydrogel, a protein hydrogel, a nucleic acid gel, a biohydrogel and, more particularly, poly(N-vinyl caprolactam) (PVCL), polyvinyl pyrrolidone (PVP), polydopamine, and a polymeric acrylic.

6. The ionic conducting gel according to Clause 5, wherein the polymeric adhesive material is selected from one or more of the group consisting of poly(N-vinyl caprolactam) (PVCL), polyvinyl pyrrolidone (PVP), polydopamine, and a polymeric acrylic.

7. The ionic conducting gel according to Clause 6, wherein the polymeric adhesive material is poly(N-vinyl caprolactam) (PVCL).

8. The ionic conducting gel according to any one of the preceding clauses, wherein the polymeric adhesive material represents about 10 wt % of the total weight of the gel.

9. The ionic conducting gel according to any one of the preceding clauses, wherein the metal salt is selected from one or more of the group consisting of a sodium salt, a potassium salt, a calcium salt, and a magnesium salt.

10. The ionic conducting gel according to Clause 9, wherein metal salt is sodium chloride.

11. The ionic conducting gel according to any one of the preceding clauses, wherein the metal salt is represents about 5 wt % of the total weight of the gel.

12. A flexible electrode device, comprising:
a flexible substrate material having a first surface and a second surface;
a metal electrode pad on the first surface of the substrate; and
an ionic conducting gel layer that covers the metal electrode pad, wherein the ionic conducting gel is as described in any one of Clauses 1 to 11.

13. The electrode device according to Clause 12, wherein the device further comprises two or more metal electrode pads on the first surface of the substrate,
where each of the two or more metal electrode pads is electrically connected to at least one conductive pathway,
where each of the two or more metal electrode pads is covered by an ionic conducting gel layer, and
where each conductive pathway is covered by an insulating material.

14. The electrode device according to Clause 12 or Clause 13, wherein the impedance of the electrode device is approximately from 10⁻³ to 10⁵Ω at from 1 to 10⁶ Hz.

15. The electrode device according to Clause 13, wherein the two or more metal electrode pads is from 2 to 512, such as 8 to 256, such as from 15 to 128 metal electrode pads.

16. A prosthetic control system comprising one or more electrode devices according to Clauses 12 to 15.

### DRAWINGS

FIG. 1 depicts schematics showing capacitive coupling of (a) surface interface and (b) inner interface. For the surface without anchoring, induced charge at the metal layer depends on transmission efficiency of EDL-1 (interface of metal layer and ionic conducting gel layer) and EDL-2 (interface of gel and stratum corneum). With anchoring, the induced charge at the metal layer increases. (c) depicts that PVCL electrodes that anchor to the SC show ~93% lower impedance than alginate polyacrylamide (Alg-PAAm) electrodes that do not anchor. Error bars in (c) are standard deviations from 10 independent experiments.

FIG. 2 depicts molecular dynamics (MD) simulation of gel electrodes interacting with the stratum corneum. (a) Schematic showing an ionic conducting gel electrode embedded into the stratum corneum—topmost layer of the skin (left). Simulation of the stratum corneum (modelled as a lipid bilayer using free fatty acid (FFA), long-chain ceramides (CER), cholesterol (CHOL)) and ionic gel electrode interface (middle) show hydrophobic molecules interact intimately with the lipid matrix (right). Due to the molecular anchoring of the gel, the coupling region was increased for the higher coupling efficiency. (b) The changes of potential mean force (PMF) along with the permeating depth. Here, we defined z=0 is the centre of a lipid matrix. From the MD results, an energy barrier ΔG should be overcome when the anchoring-molecules anchored to the lipid matrix. And this ΔG is depended on the hydrophobicity of the molecules. (c) Molecular simulation shows poly (N-vinyl caprolactam) (PVCL) interacted most with the lipid matrix amongst the 5 monomers (PVCL, polyvinylpyrrolidone (PVP), acrylic, dopamine (DOPA) and alginate). To accelerate structure evolution, simulations were done with V-rescale algorithm at 310 K, 340 K and 360 K. Evolution at 310 K and 360 K at 100 ns are shown.

FIG. 3 depicts schematic for measuring the impedance of the electrode on skin. The distance of the two electrodes (edge) is 1 cm, and the size of the electrode is 0.5 by 0.5 cm (approval number: SIAT-IRB-180315-H0242).

FIG. 4 depicts a graph that shows the systematic bioelectric impedance of commercial electrode (from Heal Force Bio-Meditech Holding Limited) and PVCL electrode on amputee skin from 1 Hz to 10⁵ Hz. The impedance at 1 Hz of PVCL electrode is nearly 1/100^{th} of commercial acrylic-based electrode.

FIG. 5 depicts molecular structure of lipid matrix. (a) Computer-generated structures of lipids ceramides (CER), cholesterol (CHOL), and free fatty acid (FFA). (b) Simulation showing monomer molecules anchored to the lipid matrix.

FIG. 6 depicts PMF and ΔG of five monomers when interacting with SC. (a)-(e): PMF calculations for (a) PVCL, (b) PVP, (c) acrylic, (d) DOPA, and (e) alginate monomers. (f) Of the 5 monomers, PVCL has the lowest energy barrier (6.57 kBT) when anchored inside the lipid matrix. Alginate monomer has the highest energy barrier (ΔG=40.67 kBT) and will not easily anchor to the lipid matrix.

FIG. 7 depicts molecular structure evolution of five monomers during the anchoring process. Simulation at high temperatures (310 K to 360 K) accelerates the monomer-lipid interaction. Of the five monomers, PVCL anchored deepest to the lipid matrix.

FIG. 8 depicts interaction of five monomers with SC over time. At 0 ns, no interaction occurred between the monomers and SC. All monomers except alginate gradually permeated into the SC over 100 ns, with PVCL showing the fastest permeation. Simulation was done at 360 K.

FIG. 9 depicts dynamic permeating process of five gels into biomimetic skin. (a)-(c) The details of order parameter, S_{z}, or the lipid matrix depended on time at (a) 310 K, (b) 340 K and (c) 360 K. Inset of (a) describe the choosing method of angle θ which decides the value of S_{z}. Clearly, S_{z} of all gels and control is about 0.65 at 310 K, means the permeating process is not fast process. When the temperature increasing to 340 K, S_{z} of PVCL gradually reaches to 0 with time increasing to 100 ns, suggests PVCL have stronger interacting with SC matrix than other 4 monomers. For the alginate, the S_{z} is always similar to the control results even when temperature enhanced to 360 K. Thus, alginate is hard to penetrate into SC. (d)-(e) Confocal microscopy images show PVCL anchored to the biomimetic skin at a depth of 3.07 μm (d) while no anchoring was seen with alginate (e). Biomimetic skin was dyed with rhodamine 6G and gels were dyed with Alexa 488. (f) Quantitative measurements show PVCL anchored most deeply to the biomimetic skin amongst the 5 monomers. Error bars are standard deviations based on 10 independent samples.

FIG. 10 depicts histogram of order parameter (S_{z}) of the lipid matrix for different monomers at 310 K, 340 K and 360 K. Consistent with PMF calculations in FIG. 6, PVCL has the lowest S_{z} at 340 K and 360 K, indicating that it disrupted the lipid structure the most. Error bars are standard deviations of S_{z} values in the last 20 ns of the simulations.

FIG. 11 depicts confocal microscopy images of PVP, acrylic and DOPA ionic conducting gels anchoring to the SC matrix. Dotted lines show the depth at which gels were embedded into the SC (biomimetic skin based on fibroin). All gels except alginate anchored to the SC matrix. PVCL anchored the deepest at 3 μm.

FIG. 12 depicts electrical properties of the five gel electrodes. (a) Photograph (left), and schematic (right) of a gel electrode composed of polydimethylsiloxane (PDMS) substrate and insulator, and Cr/Ag/Au metal contacts. (b) Adhesive force of different electrodes obtained from a standard 90° peel-off test using biomimetic skin. Consistent with simulation studies, PVCL electrode shows the highest (~189 N/m) adhesion force. (c) Graph shows the impedance of various electrodes on human skin from 1 Hz to 10⁵ Hz. (d) Comparing the impedance and adhesion force of our PVCL electrode with different electrodes: 1: CNT/aPDMS (Lee, S. M. et al., Sci. Rep., 2014, 4, 6074), 2: PDMS_40 NW (He, K. et al., ACS Nano, 2022, 16, 9691), 3: PDMS_40 NW/Tape (Kim, J.-H. et al., Nano Lett., 2018, 18, 5431), 4: Au/Parylene (Nawrocki, R. A. et al., Adv. Funct. Mater., 2018, 28, 1803279), 5: Silk (Chen, G. et al., Adv. Mater., 2018, 30, 1800129), 6: commercial electrodes (from Heal Force Bio-Meditech Holding Limited), 7: silk/Au (Chen, G. et al., Adv. Mater., 2018, 30, 1800129), 8: Fe@Sibione (Jang, K. I. et al., Adv. Funct. Mater., 2016, 26, 7281), 9: PDA-rGO-PAM (Han, L. et al., Small, 2017, 13, 1601916), 10: a4-PDMS_40 NW (Kim, J.-H. et al., Nano Lett., 2018, 18, 5431), 11: Alg-PAAm (Pan, L. et al., Adv. Mater., 2020, 32, 2003723). Impedance decreased with more adhesive electrodes (curve). Shaded area shows intrinsic impedance of human skin. Systematic impedance (total impedance of skin, electrode and interface) of PVCL electrode on skin (14 kΩ) approaches the impedance of human skin (6-10 kΩ). (e) Recording limit of PVCL electrode (~1.5% MVC) on human skin approaches the lower limit of human muscular contraction (~1% MVC). For muscle contraction below 10% MVC, signal losses (shaded area) occurred with DOPA, acrylic, and alginate electrodes (all the data recorded under same amplification of 24, the Y axle is relative intensity and the curve was shifted about 5 mV.). (f) SNR of PVCL electrodes was best (>5) amongst the 5 electrodes when recording low-level (1.7% MVC) myoelectric signals on human skin. Traditional recognition systems require a SNR>5. Error bars are standard deviations based on 10 independent samples (all data recorded under same amplification of 24).

FIG. 13 depicts 90° peel-off test of PVCL, PVP, Alginate, Acrylic and DOPA on biomimetic skin and it shows adhesion of PVCL is better than others. Error bar are the standard deviations based on 10 independent samples.

FIG. 14 depicts 180° peel-off test of PVCL, PVP, Alginate, Acrylic and DOPA. Similarly. PVCL is most adhesive in the five gels. This result agreed well with that of a 90° peel-off test shown in FIG. 9b and FIG. 13.

FIG. 15 depicts impedance of electrodes based on five molecular anchors. (a) Intrinsic impedance of the five ionic conducting gels for 1 to 104 Hz. (b) Systematic bioelectric impedance of five electrodes. Error bars are standard deviations from 20 samples for the gel electrodes.

FIG. 16 depicts dexterous myoelectric control. (a) Photograph of our myoelectric prosthetic control system on an amputated arm. Gel electrodes attached on residual active muscles are connected to a wireless data recording box via a flexible cable. Size of electrode array depends on size of muscle being measured. (b) A real-time pixel image (left; size 1×16×200 ms) of myoelectric signals obtained from the amputee's extensor carpi ulnaris muscle using PVCL electrode array with n=16 channels. The pixel image is input into a convolutional neural network (CNN)-based image recognition architecture (right) that extracts and processes the features, and classifies them according to the finger and wrist movements initiated by the amputated limb. (c) A myoelectric pixel image (left) subjected to the mean average value (MAV) attention block (rectangle box in the middle) shown in (b) returns a featured mapping (right) that can prejudge a real-time myoelectric map and theoretically reduce recognition time from 200 ms to 20 ms. (d) Confusion matrix for G1 to G6 finger and wrist movements at low-level (≤20% MVC) muscle contraction obtained using PVCL electrodes show the CNN algorithm is highly accurate (97.6%). Row represents actual classification for G1-G6 while column is the predicted classification. Values of diagonal elements represent the degree of correctly predicted classes. Reset is myoelectric signals without finger and wrist movements. (e) Recognition accuracy of CNN for both low-level (≤20 MVC; CNN-L) and high-level (≥20 MVC; CNN-H) signal. (f) Photographs shows the myoelectric control of a below-elbow amputee driving the arm of a robotic car (prosthesis) to grasp and place a candle (with diameter of 1.5 cm) into a hole (with diameter of 2 cm). The signal source of the control was the real-time myoelectric mapping generated from G1-G6.

FIG. 17 depicts myoelectric mappings of finger and wrist movements initiated by an amputated limb. Photograph (left column) of an amputee with our myoelectric control system attached on the amputated left arm. Type of finger and wrist movement (G1-G6) initiated by the amputated limb (red dotted line) is shown on the right arm as reference. Pixel images of myoelectric signals from 100% MVC, 20% MVC (high level), s 20% MVC (low-level) and the corresponding featured mapping (right column) for each movement are used to drive the prosthesis (the arm of a robotic car in our study) to release (G1), grasp (G2), move left (G3), move right (G4), move upwards (G5) and downwards (G6). Myoelectric map of 100% MVC is used as the source to preliminarily extract the active electrode and weight of G1-G6 through the attention block. Featured mappings are the extracted results that can be used as an initial judgement for real-time myoelectric mapping. Theoretically, prejudgement can decrease the delay time of the recognition process from 200 ms to 20 ms. High and low-level myoelectric mappings are used as the source for the next fine-tuning step to further improve the classification accuracy. Grey level of the myoelectric mapping is relative intensity of the muscle contraction.

FIG. 18 depicts a unit dataset of (a) high-level (≥20% MVC) and (b) low-level (≤20% MVC) of G1 for training. The time of the unit dataset is 2 s. To extract the feature, the dataset will be divided to 10 with every package about 200 ms.

FIG. 19 depicts a unit dataset of (a) high-level (≥20% MVC) and (b) low-level (≤20% MVC) of G2 for training. The time of the unit dataset is 2 s. To extract the feature, the dataset will be divided to 10 with every package about 200 ms.

FIG. 20 depicts a unit dataset of (a) high-level (≥20% MVC) and (b) low-level (≤20% MVC) of G3 for training. The time of the unit dataset is 2 s. To extract the feature, the dataset will be divided to 10 with every package about 200 ms.

FIG. 21 depicts a unit dataset of (a) high-level (≥20% MVC) and (b) low-level (≤20% MVC) of G4 for training. The time of the unit dataset is 2 s. To extract the feature, the dataset will be divided to 10 with every package about 200 ms.

FIG. 22 depicts a unit dataset of (a) high-level (≥20% MVC) and (b) low-level (≤20% MVC) of G5 for training. The time of the unit dataset is 2 s. To extract the feature, the dataset will be divided to 10 with every package about 200 ms.

FIG. 23 depicts a unit dataset of (a) high-level (≥20% MVC) and (b) low-level (≤20% MVC) of G6 for training. The time of the unit dataset is 2 s. To extract the feature, the dataset will be divided to 10 with every package about 200 ms.

### DESCRIPTION

As noted above, it has been surprisingly found that the generation and use of an ionic conducting gel in the production of gel electrodes that firmly anchor into the skin reduce the barrier effect noted above and increase the ionic-electronic coupling regions.

Thus, in a first aspect of the invention, there is provided an ionic conducting gel that comprises:
a polymeric adhesive material in an amount of from 5 to 20 wt % of the gel;
a metal salt in the amount of from 2 to 10 wt % of the gel; and
the balance being a hydrophilic polymeric material, wherein a gel matrix is formed by between the polymeric adhesive material and the hydrophilic polymeric material, and the metal salt is distributed throughout the gel matrix.

In embodiments herein, the word “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

The phrase, “consists essentially of” and its pseudonyms may be interpreted herein to refer to a material where minor impurities may be present. For example, the material may be greater than or equal to 90% pure, such as greater than 95% pure, such as greater than 97% pure, such as greater than 99% pure, such as greater than 99.9% pure, such as greater than 99.99% pure, such as greater than 99.999% pure, such as 100% pure.

As used herein, the singular forms “a,” “an,” and “the” include plural referents unless the context clearly dictates otherwise. Thus, for example, reference to “a composition” includes mixtures of two or more such compositions, reference to “an oxygen carrier” includes mixtures of two or more such oxygen carriers, and the like.

Without wishing to be bound by theory, it is believed that the gel matrix may be formed by intermolecular interactions between the polymeric adhesive material and the hydrophilic polymeric material. Such intermolecular interactions may be in the form of hydrogen bonding, Van Der Waal's interactions and the like. As will be appreciated, the gel matrix may be formed by the mixing of the polymeric adhesive material and the hydrophilic polymeric material and may form a gel even if one (or both) of the components is originally in a liquid phase at standard temperature and pressure.

In embodiments of the invention that may be mentioned herein, the number average molecular weight of the hydrophilic polymeric material may be from 200 to 15,000 Daltons. For example, the number average molecular weight of the hydrophilic polymeric material may be 200 Daltons. For the avoidance of doubt, the term “Daltons” is used herein to represent atomic mass units.

Any suitable hydrophilic polymeric material may be used in embodiments of the current invention. Examples, of suitable hydrophilic polymeric materials include, but are not limited to, polyethylene glycols and polyalcohols. In particular embodiments of the current invention, the hydrophilic polymeric material may be a polyethylene glycol. For example, the polyethylene glycol may have a number average molecular weight of 200.

Any suitable polymeric adhesive material may be used in embodiments of the current invention. Examples, of suitable polymeric adhesive materials include, but are not limited to, polysaccharides, cellulose hydrogels, protein hydrogels, nucleic acid gels, biohydrogels and, more particularly, poly(N-vinyl caprolactams) (PVCL), polyvinyl pyrrolidones (PVP), polydopamines, polymeric acrylics and combinations thereof. In more particular embodiments of the invention that may be mentioned herein, the polymeric adhesive material may be selected from one or more of the group consisting of poly(N-vinyl caprolactam) (PVCL), polyvinyl pyrrolidone (PVP), polydopamine, and a polymeric acrylic. For example, in certain embodiments of the invention that may be mentioned herein, the polymeric adhesive material may be poly(N-vinyl caprolactam) (PVCL).

Any suitable amount of the polymeric adhesive material may be used in embodiments disclosed herein, such as from 2 to 10 wt %, such as from 3 to 5 wt % and the like. In particular embodiments of the invention, the polymeric adhesive material may represent about 10 wt % of the total weight of the gel.

The metal salt used in the ionic conducting gel may be any suitable metal salt. Examples of suitable metal salts include, but are not limited to, iron salts or, more particularly, sodium salts, potassium salts, calcium salts, magnesium salts, and combinations thereof. Any suitable counterion to the metal may be used, for example the counterion may be one or more of the group consisting of fluoride, iodide, chloride, bromide, phosphate, sulfate and the like. Particular salts that may be mentioned include the kali (or tissue) salts. It is noted that the kali salts are generally considered to be found in the body and may therefore be considered compatible with the desired use of the ionic conducting gel. For example, iron phosphate or, more particularly, calcium fluoride, calcium phosphate, potassium phosphate, potassium sulfate, and sodium. In particular embodiments of the invention, the metal salt may be sodium chloride.

As will be appreciated, the purpose of the metal salt in the ionic conducting gel is to enhance the conductivity of the gel. As such, other materials that may be substituted to replace the metal salt are explicitly contemplated herein.

Any suitable amount of metal salt may be present in the ionic conducting gel. For example, the metal salt may represents from 5 to 20 wt % of the total weight of the gel, such as from 5 to 10 wt % of the total weight of the gel, such as about 5 wt % of the total weight of the gel.

The ionic conducting gel disclosed herein has been found to be particularly suitable for use in the formation of flexible electrode devices that may be used in a prosthetic device. Hence, in a second aspect of the invention, there is provided a flexible electrode device, comprising:
a flexible substrate material having a first surface and a second surface;
a metal electrode pad on the first surface of the substrate; and
an ionic conducting gel layer that covers the metal electrode pad, wherein the ionic conducting gel is as described hereinabove.

Any suitable material may be used as the flexible substrate material. For example, the flexible substrate material may be a suitable elastomer, such as polydimethylsiloxane (PDMS). As will be appreciated, the flexible substrate material may act as an electrical insulator material too.

The metal electrode pad may be provided on a surface of the flexible substrate material (e.g. the first surface) and may be formed from any suitable conductive material or combination of such materials. For example, the metal electrode pad may be formed from layers of chromium, silver and gold, where the chromium forms the base layer attached to the flexible substrate material and gold forms the top layer.

In embodiments of the second aspect of the invention, the metal electrode pad may be connected to a conductive pathway (e.g. a wire, such as a gold wire). In further embodiments of the invention, the metal electrode pad and, when present, the conductive pathway may be covered by an insulating material.

In embodiments of the second aspect of the invention, the device may further comprise two or more metal electrode pads on the first surface of the substrate,
where each of the two or more metal electrode pads is electrically connected to at least one conductive pathway,
where each of the two or more metal electrode pads is covered by an ionic conducting gel layer, and
   where each conductive pathway is covered by an insulating material. Any suitable number of metal electrode pads may be used herein. For example, there may be from from 2 to 512, such as 8 to 256, such as from 15 to 128 metal electrode pads.

The insulating material used herein may be any suitable insulating material that displays a suitable degree of flexibility. For example, the insulating material may be PDMS.

The flexible electrode devices disclosed herein may display reduced levels of impedance relative to conventional flexible electrode devices intended for use in a prosthetic control system and/or prosthetic device. For example, the impedance of the flexible electrode device may be approximately from 10⁻³ to 10⁵Ω at from 1 to 10⁶ Hz. For example, the flexible electrode device may have an impedance of from 0.1 to 50 kΩ at from 1 to 10⁶ Hz, such as about 20 kΩ at from 1 to 10⁶ Hz.

The manufacture of the flexible electrode device may be conducted using any suitable technique known in the art using the materials disclosed herein. Details of how such a flexible electrode device may be manufactured and applied to a prosthetic control system (and hence to a prosthetic device) are provided in the experimental section below.

In yet a further aspect of the invention, there is provided a prosthetic control system comprising one or more electrode devices as described hereinbefore. The manufacture of the prosthetic control system may be conducted using any suitable technique known in the art using the materials disclosed herein. Details of how such a control system may be manufactured and applied to a prosthetic device are provided in the experimental section below.

Compared to the traditional improvement of skin-electrode surface physicochemical properties, we have discovered that the use of a gel that can employ molecular anchoring enables the gel electrode to embed into the stratum corneum to weaken the barrier effect of skin and increase the ionic-electronic coupling interface for higher efficiency. For example, as described in the examples below, a gel electrode material that uses poly(N-vinyl caprolactam) as the polymeric adhesive material may provide a gel electrode that has an impedance of only about 20 ko (which is about 1/100 the impedance of commercial electrodes) with amputees' skin and can detect the ultralow myoelectric signals to 1.5% MVC, approaching human limitation. This molecule anchoring-based electrode paves a new way for dexterous myoelectric control.

It is believed that the inclusion of a polymeric adhesive material allows for molecular anchoring into the skin provides the good effects noted above and in the examples. The polymeric adhesive materials used herein may each have a high adhesive force with skin (e.g. from 50 to 250 N/m, such as from 75 to 200 N/m, such as from 100 to 190 N/m, such as about 189 N/m in a standard 90° peel-off test using bimimetic skin).

Further aspects and embodiments of the invention are discussed in the following lettered clauses.
A) Conducting gels for molecule anchoring, comprising:
(a) 5% NaCl to improve the ionic conducting electrode preparation; and
(b) PVCL, PVP, dopamine and acrylic were dissolved in PEG (200) with 10% wt %.

Alginate gel was done in one step, by mixing water with sodium alginate, ionic cross-linker, covalent cross-linker, acrylamide, and thermosinitiator as described in the examples below.
B) A method of fabricating a electrode with ultralow bioelectronic impedance, comprising:
a) to create a PDMS substrate, mix liquid PDMS with the PMDS curing agent with a ratio of 10:1. The mixture is then stirred until it is homogeneous by using a centrifugal mixer;
b) after the mixture is homogeneous, it is put inside the vacuum chamber to eliminate bubbles. It is then followed by the deposition of the PDMS layer on top of fluorinated silicon wafers;
c) the fluorinated wafers are made by immersion of silicon wafer to a 10 mM solution of fluoroalkyl silane (heptadecafluoro-1,1,2,2-tetrahydro decyl trithoxysilane) in toluene for 30 minutes, followed by a heat treatment at 150° C. for 1 hour;
d) the deposition of PDMS is done by spin coating at 1000 rpm on top of fluorinated wafers for 40 seconds. The final step is to let it cure at 60° C. for 4 hours;
e) deposition of the conducing layer Cr/Ag/Au on top of the PDMS substrate is done by the sputtering technique;
f) before the gold layer is sputtered, a 5 nm Chromium layer is deposited on top of PDMS to increase the adhesion between PDMS and the gold layer for better contact;
g) a silver layer with a thickness of 400 nm is sputtered by using a sputtering machine;
h) a gold layer with 20 nm is deposited on the sliver to prevent oxidation; and
i) on top of the gold layer, a PDMS is deposited as an insulating layer to prevent the gold connected to the device from collecting any signal from the surface of the skin.

Further aspects and embodiments of the invention will now be discussed by reference to the following non-limiting examples.

### EXAMPLES

### Materials

All reagents were purchased from commercial sources and used without further purification.

### Example 1. Molecule Anchoring of Poly (N-Vinyl Caprolactam) (PVCL) Gel Electrodes to the Skin for Ultralow Myoelectric Signal Monitoring

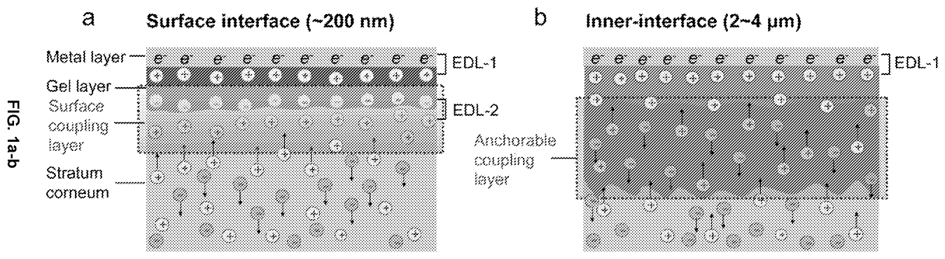

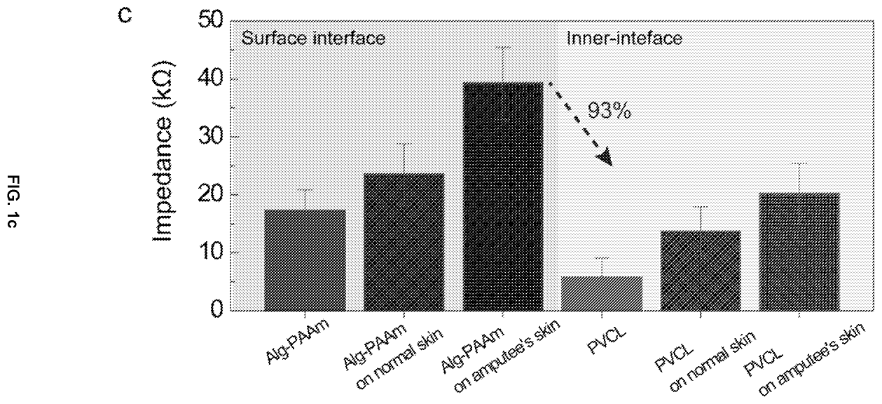

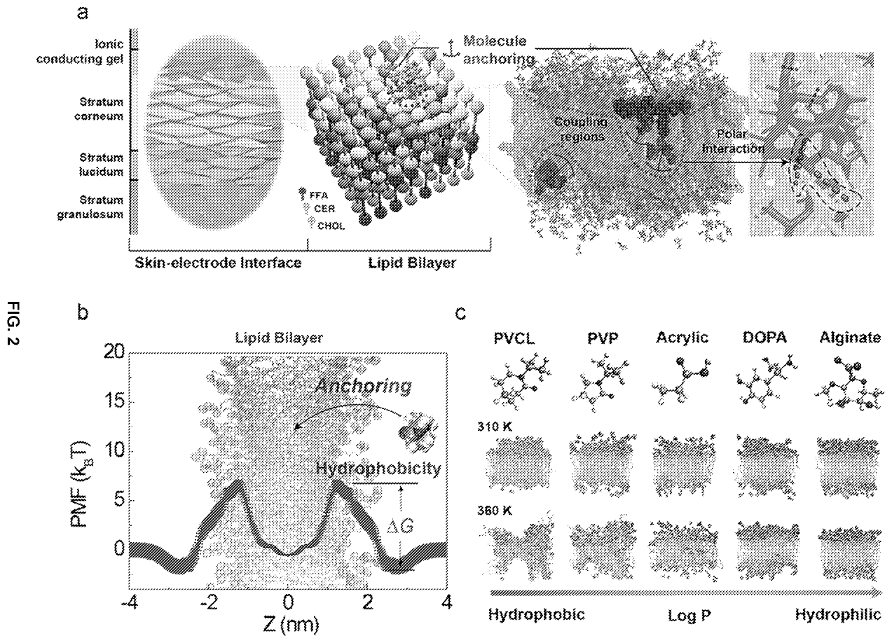

We designed hydrophobic poly (N-vinyl caprolactam) (PVCL) gel electrodes that firmly anchor to the skin to reduce the barrier effect and increase the ionic-electronic coupling regions, as shown in FIG. 2a and FIG. 1b.

### Electrode Fabrication Methods: PDMS Substrate Fabrication

To create a PDMS substrate, liquid PDMS with the PDMS curing agent were mixed with a ratio of 10:1. The mixture was then stirred until it is homogeneous by using a centrifugal mixer. After the mixture was homogeneous, it was put inside the vacuum chamber to eliminate bubbles. It was then followed by the deposition of the PDMS layer on top of fluorinated silicon wafers. The fluorinated wafers were made by immersion of silicon wafer to a 10 mM solution of fluoroalkyl silane (heptadecafluoro-1,1,2,2-tetrahydro decyl trithoxysilane) in toluene for 30 minutes, followed by a heat treatment at 150° C. for 1 hour. The purpose of fluorination was to create a hydrophobic surface. The deposition of PDMS was done by spin coating at 1000 rpm on top of fluorinated wafers for 40 seconds. The final step was to let it cure at 60° C. for 4 hours.

### Electrode Fabrication Methods: Deposition of Cr/Ag/Au and Insulator

Deposition of the conducing layer Cr/Ag/Au on top of the PDMS substrate was done by the sputtering technique (by method disclosed in Pan, L. et. al., Adv. Mater., 2020, 32, 2003723). A very thin layer (in the scale of nm) was needed to preserve its compliance ability to conduct even when it was stretched.

Before the gold layer was sputtered, a 5 nm chromium layer was deposited on top of PDMS to increase the adhesion between PDMS and the gold layer for better contact. Then, a silver layer with a thickness of 400 nm was sputtered by using a sputtering machine. Finally, a gold layer with 20 nm was deposited on the sliver to prevent oxidation. A mask was used in the sputtering process to control and pattern the deposition of gold in the desired area. Next, on top of the gold layer, a PDMS was deposited as an insulating layer to prevent the gold connected to the device from collecting any signal from the surface of the skin.

### Electrode Fabrication Methods: Ionic Conducting Gels Fabrication

For all gels, we introduced 5% NaCl to improve the ionic conductivity. PVCL, PVP, dopamine and acrylic were dissolved in polyethylene glycol (PEG) 200 with 10% wt %. Alginate gel was done in one step, by mixing water with sodium alginate, ionic cross-linker, covalent cross-linker, acrylamide, and thermosinitiator according to our previous work (Pan, L. et. al., Adv. Mater., 2020, 32, 2003723).

It is noted that after dissolution of the PVCL, PVP, dopamine and acrylic polymeric materials into the PEG 200 that a gel was formed.

### Systematic Impedance of Electrode and Skin

The systematic impedance was measured through Zahner scientific instruments. During testing, two electrodes with a size of 1×1 cm² were pasted on the skin at a distance of ~1 cm. The frequency ranged from 1 Hz to 10⁵ Hz.

### Results and Discussion

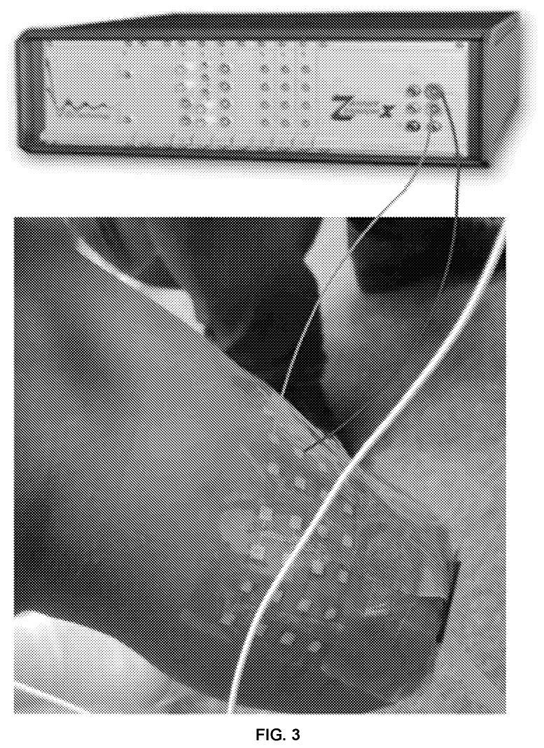

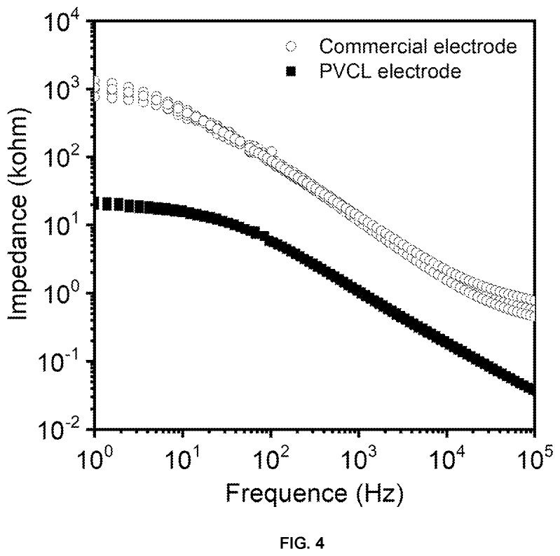

The increased coupling regions lead to a higher probability of coupling between ionic fluxes and electronic currents, enhancing the ionic-electronic coupling efficiency. Our measurements (FIG. 3) on amputee's skin showed that the impedance of the PVCL electrode is approximately 20 kΩ@1 Hz, which is nearly 1/100 of the impedance of commercial acrylic-based electrodes (FIG. 4) and 93.4% lower than that of alginate polyacrylamide (Alg-PAAm) electrodes (Pan, L. et al., Adv. Mater., 2020, 32, 2003723), which showed the lowest bioelectronic impedance on normal skin (FIG. 1c). This suggests that PVCL gel electrode can be used to monitor ultralow (down to 1.5% MVC) myoelectric signals for the amputees on the dexterously prosthetic control.

### Example 2. Molecular Dynamics (MD) Simulation of Molecule Anchoring Process of PVCL, Polyvinylpyrrolidone (PVP), Acrylic, Dopamine (DOPA) and Alginate

Inspired by these measurements, we performed molecular dynamics (MD) simulation to understand how PVCL molecules interact with and anchor to the SC. We also examined 4 other adhesive molecules: polyvinylpyrrolidone (PVP), acrylic, dopamine (DOPA) and alginate, to explain the molecular anchoring strategy.

### Modelling of Structures of Monomers

We studied five types of molecules, i.e., the monomers of gels of PVCL, PVP, acrylic, dopamine (DOPA), and alginate, as shown in FIG. 1b. The structures of the monomers were obtained by the CHARMM-GUI web service (https://www.charmm-qui.org/).

### Modelling of Surface Layer of Skin

According to previous studies, the skin's surface layer, i.e., the stratum corneum (SC), was modelled by the lipid matrix of SC (Gupta, R. et al., J. Phys. Chem. B, 2016, 120, 8987).

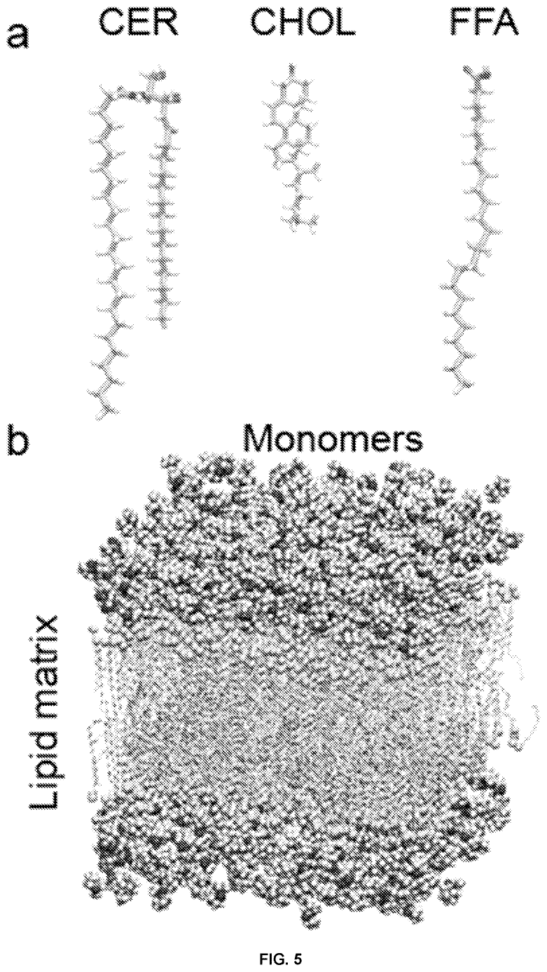

The lipid matrix of SC was constructed by the CHARMM-GUI web service using a heterogeneous mixture of long-chain ceramides (CER), cholesterol (CHOL), and free fatty acid (FFA) at a 1:1:1 ratio according to previous studies, as shown in FIG. 2a and FIG. 5 (Harding, C. R., Dermatol. Ther., 2004, 17, 6; Notman, R. & Anwar, J., Adv. Drug Deliv. Rev., 2013, 65, 237). The lipid matrix was constructed as a lipid matrix in the size of approximate 10×10 nm². To determine the interaction between the monomers and the SC, 700 monomers were added randomly to the simulation box, as shown in FIG. 5.

### Molecular Dynamics Simulation Methods

The molecular dynamics (MD) simulations were performed by GROMACS package (Van Der Spoel, D. et al., J. Comput. Chem. 2005, 26,), with CHARMM36 force field (Klauda, J. B. et al., J. Phys. Chem. B, 2010, 114, 7830). TIP3P (Jorgensen, W. L. et al., The J. Chem. Phys., 1983, 79, 926) water molecules were used to solvate the system, and an appropriate number of Na⁺ ions were added to neutralize the system. The simulations were performed in the periodic boundary condition. All the graphics and visualization analyses were performed by the Visual Molecular Dynamics VMD package (Humphrey, W. et al., J. Mol. Graph., 1996, 14, 33).

### Results and Discussion

Corroborating with the intrinsic hydrophobicity of the lipid matrix (which consists of FFA, CER, and CHOL), the calculated potential mean force (PMF) showed that more hydrophobic monomers have a lower energy barrier (ΔG) to anchor to the lipid matrix and will embed more easily (FIG. 2b).

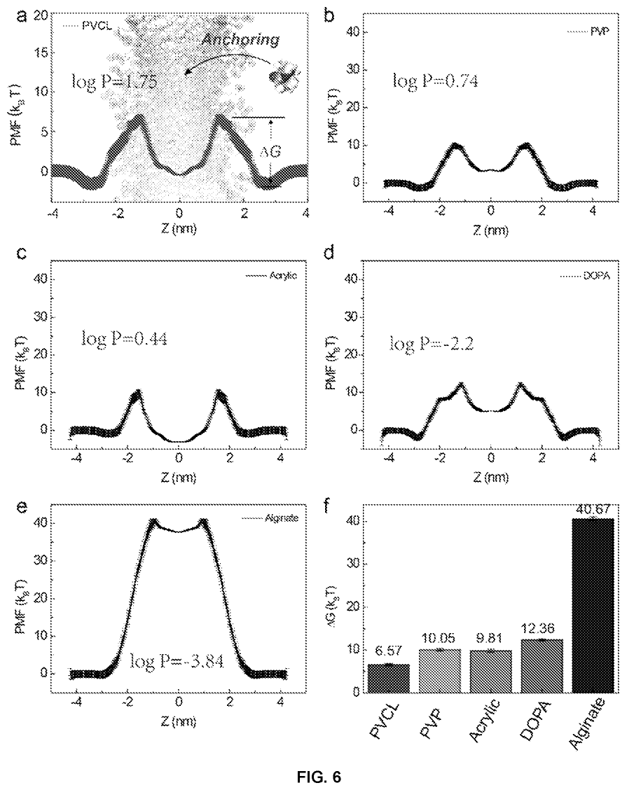

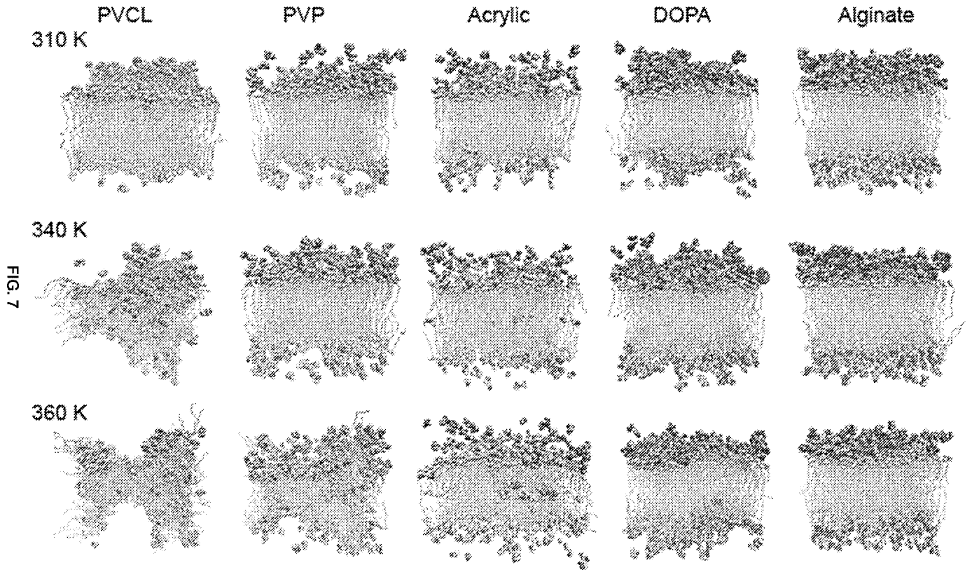

Of the five monomers, PVCL showed the lowest ΔG (6.57 k_{B}T) for anchoring to the SC because of its highest hydrophobicity. PVCL and PVP are widely used in biology and medicine and have good biocompatibility and chemical stability (Heinzmann, C. et al., Chem. Sov. Rev., 2016, 45, 342). Although PVP and PVCL have high hydrophobicity and owe homologous structure, the additional methylene group in PVCL makes it more hydrophobic than PVP. Thus, the octanol-water partition coefficient (log P) of PVCL of 1.75 is larger than that of PVP (0.74) (FIG. 6). Acrylic, DOPA and alginate are suitable adhesives and universally applied in electrodes for myoelectric recording. Log P values for acrylic (0.44), DOPA (−2.2) and alginate (−3.84) were much lower than those for PVP and PVCL. The least hydrophobic alginate displayed the highest energy barrier (ΔG=40.67 k_{B}T) and was least able to anchor to the lipid matrix.

### Example 3. Molecular Dynamics (MD) Simulations Coupled with V-Rescale Algorithm at Different Temperatures for Acceleration of the Structure Evolution

Furthermore, we investigated the interactions between the five monomers and the lipid matrix through the MD simulation coupled with the V-rescale algorithm at three temperatures, 310 K, 340 K and 360 K, respectively, to accelerate the structure evolution.

Molecular Dynamics (MD) Simulations Coupled with V-Rescale Algorithm

MD simulations were conducted by following the protocol in Example 2.

To accelerate the structure evolution, the simulations were conducted coupled with the V-rescale algorithm (Bussi, G. et al., J. Chem. Phys., 2007, 126, 014101) in three temperatures, i.e., 310K, 340K, and 360K, respectively. The systems were firstly equilibrated in an NPT ensemble, in which the pressure was coupled in a semi-isotropic condition at 1 bar with the Parrinello-Rahman method (Parrinello, M. & Rahman, A., J. Appl. Phys., 1981, 52, 7182). In the equilibrated simulations, positional restraints were applied to all the heavy atoms of the monomers and the lipids matrix. After about 1 ns equilibrium simulation, all the production simulations were performed in the NVT ensemble. The LINCS algorithm was used to restrain the covalent bonds of hydrogen atoms. The time step was set to 2 fs. The cutoff of the non-bonded interactions was set to 1.2 nm. The long-range electrostatic interaction was calculated by the particle mesh Ewald (PME) method.

### Umbrella Sampling Method

To calculate the potential of mean force (PMF) of varied monomers across the lipid matrix of the skin, the umbrella sampling method was used. In the umbrella sampling method, the reaction coordinate was defined as the distance between the centre of mass (COM) of the monomer and the lipid matrix in the normal direction, as shown in FIG. S5. To construct the structure of the system with varied reaction coordinates, the PLUMED package was applied to add the monomer to the corresponding reaction coordinate. In the umbrella sampling simulation, the size of the lipid matrix was approximate 5.4×5.4 nm². The spacing of the sampling windows was 0.1 nm. There are 41 windows were constructed for each system. In each window, 20 ns of MD simulation was performed to ensure the convergence so that a total simulation time of 800 ns was utilized for umbrella sampling in each monomer. The spring constant of the position restraints in the umbrella sampling method was 1660.5 pN/nm. Analysis of the umbrella sampling results was performed with the weighted histogram analysis method (WHAM).

### Calculation of the Order Parameter of the Lipid Matrix

To describe how the monomers influence the lipid matrix structure, we calculated the order parameter, S_{z}, of the lipid matrix as follows (Eq. (1)):

$${S}_{Z} = \frac{3}{2} < {cos}^{2} θ > - \frac{1}{2} \qquad ( 1 )$$

where θ is the angle between vectors \vec{r}₁ (the average direction of the lipid matrix) and \vec{r}₂ (the vector from Cₙ₊₁ to Cₙ₋₁₁ in the alkyl chain). The brackets <⋅> means averaging over the carbon atoms in the alkyl chains of the lipid.

In general, S_{z}=1 corresponds to a perfect alignment of the lipid in the matrix with the normal direction; S_{z}=0 indicates a random alignment of the lipid in the matrix; S_{z}=½ shows that the lipid aligns perpendicular to the normal direction of the matrix.

### Results and Discussion

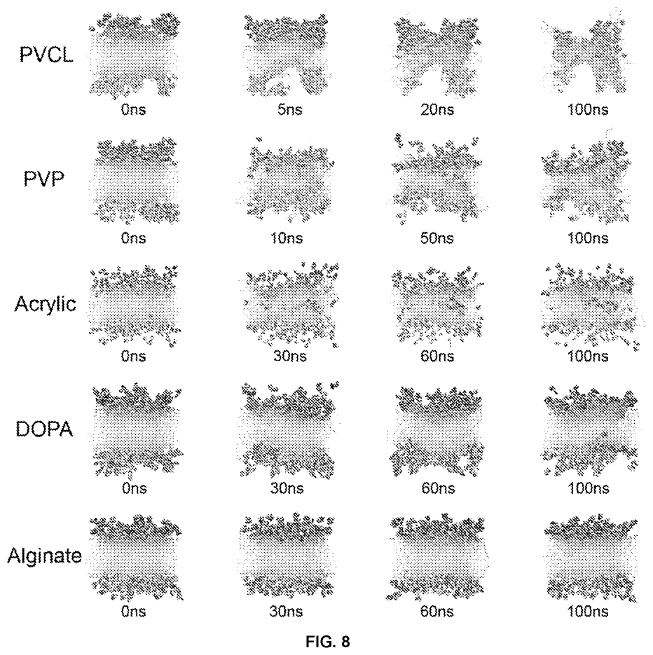

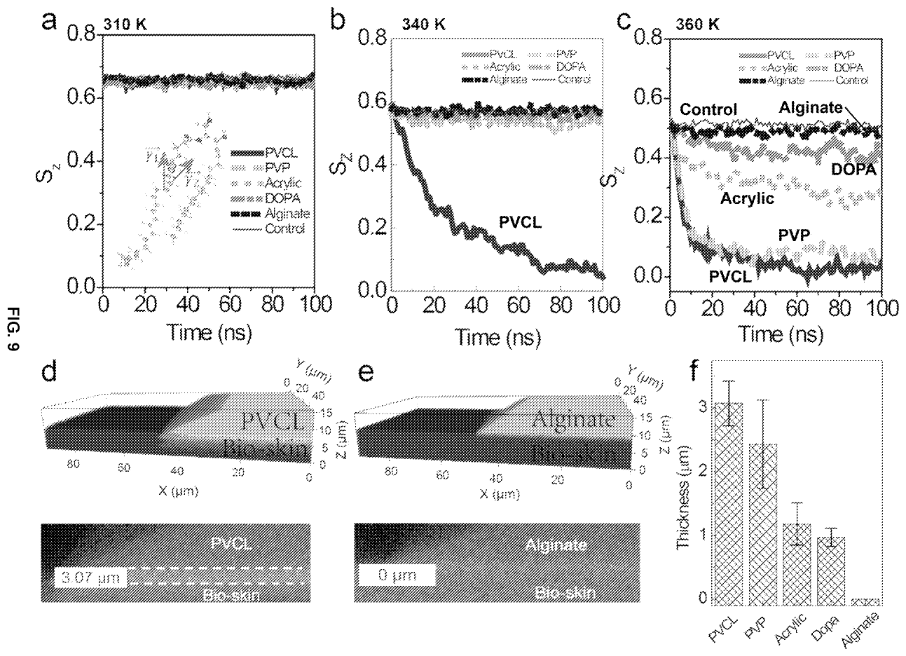

Further MD simulation coupled with the V-rescale algorithm at three temperatures (310 K, 340 K and 360 K) to accelerate the interaction also showed that PVCL embedded most effectively and fastest into the SC (FIG. 2c, FIG. 8 and FIG. 9). FIG. 2c shows the interaction between the five molecules and lipid matrix from 310 K to 360 K from 0 ns to 100 ns. The strength and depth of interaction increased with monomer hydrophobicity.

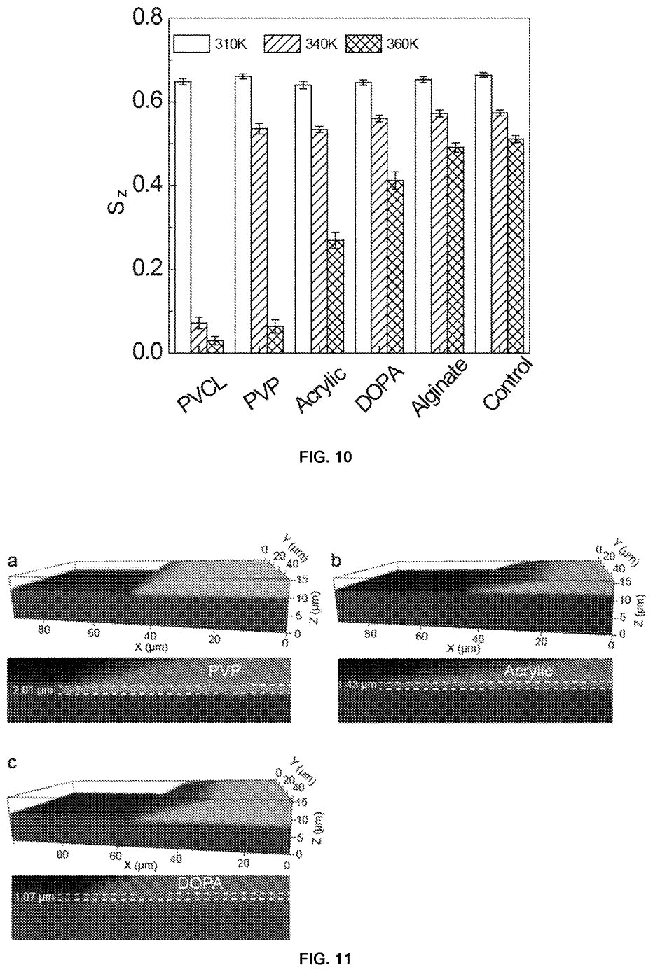

The order parameter, S_{z}, of the lipid matrix was calculated to describe how the monomers influence the lipid matrix structure. A well-ordered lipid matrix without monomers (control) had a high S_{Z} (≈0.65) over the 100 ns simulation timescale at 310 K (FIG. 9a) (see the control simulation without monomers in FIG. 10). Since the molecule anchoring process is a long-timescale process, the order parameters of the lipid matrix remained at about 0.65 in the 100 ns simulations with 310 K, similar to that without the anchoring process. When interacting with PVCL monomers at 340 K, S_{Z} decreased to 0.072 (FIG. 9b), indicating that the monomers significantly disrupted the order of the lipid matrix (FIG. 2c). For all other monomers, S_{Z} remained ≈0.55 at 340 K. When the system was heated up to 360 K, these monomers affected the lipid matrix structures differently (FIG. 9(c) and FIG. 10). As shown in FIG. 10, the simulations concluded that PVCL has the highest capacity to anchor to the skin among the five molecules.

### Example 4. Permeating Process of the Gel Electrode on the Biomimetic Skin

### Permeating Testing

Firstly, the biomimetic skin was immersed in rhodamine 6G (0.002 mg/mL, the solvent is ethanol) for 72 hours. Then, the biomimetic skin was rinsed with ethanol 3 times before drying. Secondly, the ionic conducting gels were mixed with Alexa 488 (0.004 mg/mL, the solvent is dimethylsulfoxide (DMSO)) for 24 hours. Thirdly, the dyed gels were transferred on the biomimetic skin with rhodamine 6G. We employed the confocal microscope of Leica to observe the permeating process.

### Results and Discussion

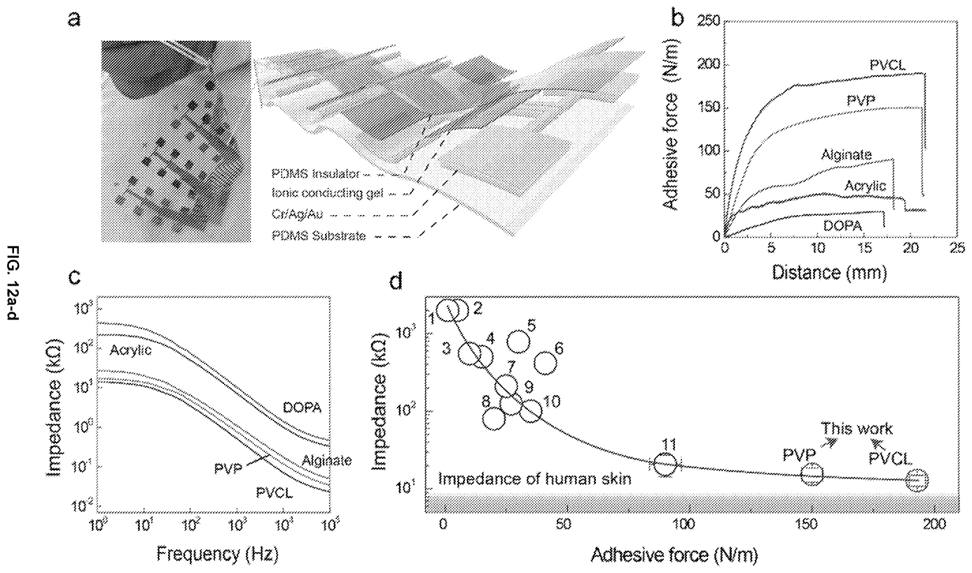

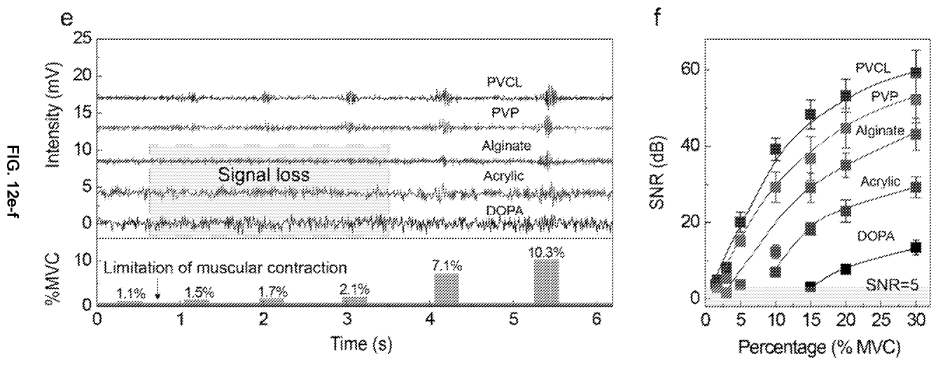

Confocal microscopy images also showed that PVCL anchored to a biomimetic skin made from fibroin at a depth of 3.07 μm, which was deeper than PVP (2.43 μm), acrylic (1.18 μm), DOPA (0.97 μm) and alginate (0 μm) (FIG. 9d-f and FIG. 11). These results demonstrated that among the 5 monomers, the most hydrophobic PVCL monomer anchored the best to the skin.

### Example 5. Fabrication of Gel Electrodes and Evaluation of the Mechanical and Electrical Properties

Based on the simulation results, we fabricated five gel electrodes using these monomers and evaluated their mechanical and electrical properties (FIG. 12).

### Fabrication of Gel Electrodes

Gel electrodes were fabricated by following the protocol in Example 1. We used PDMS as the substrate and insulator for all electrodes. To improve ionic conductivity, we added 5% NaCl to all gels.

### Equation of the Signal-to-Noise Ratio (SNR) (Eq. (2))

$$SNR = 10 {log}_{10} ( {P}_{signal} / {P}_{noise} ) \qquad ( 2 )$$

P_{signal} is the mean of pixel values
Pₙₒᵢₛₑ is the standard deviation or error value of the pixel values.

### Results and Discussion

FIG. 12a shows the structure of our electrode, and PDMS was used as the substrate and insulator of the electrodes for well complying with the curved amputees' skin.

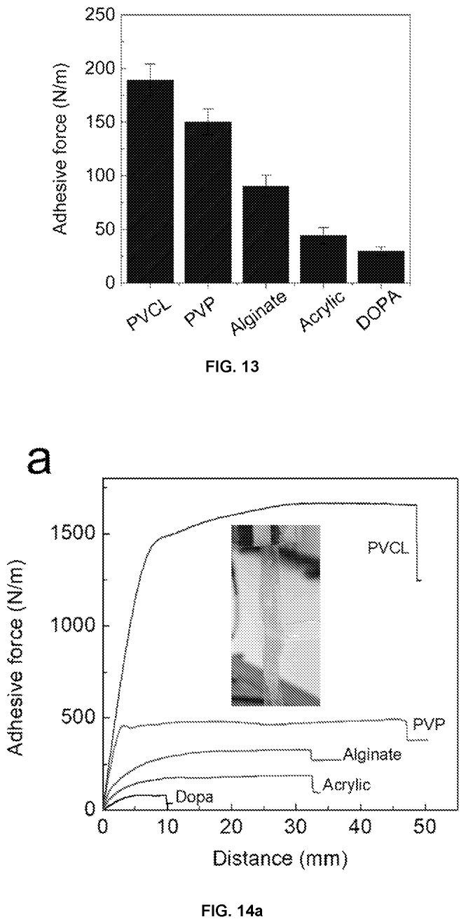

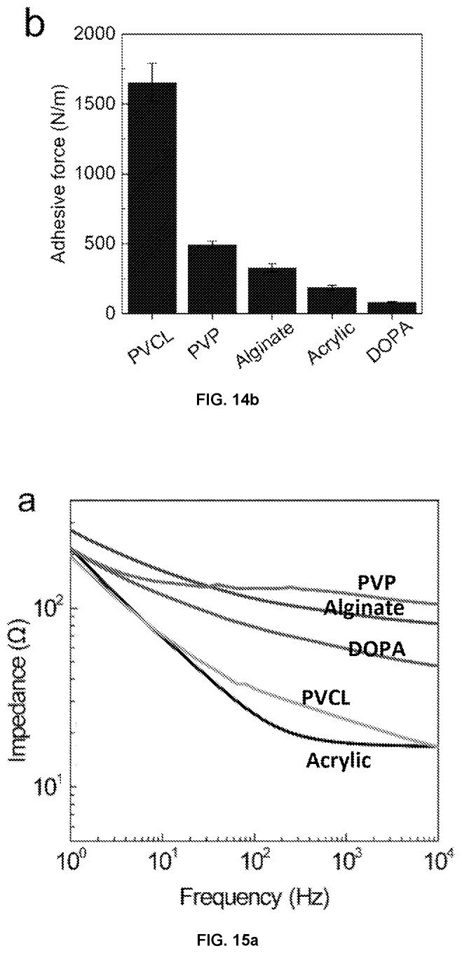

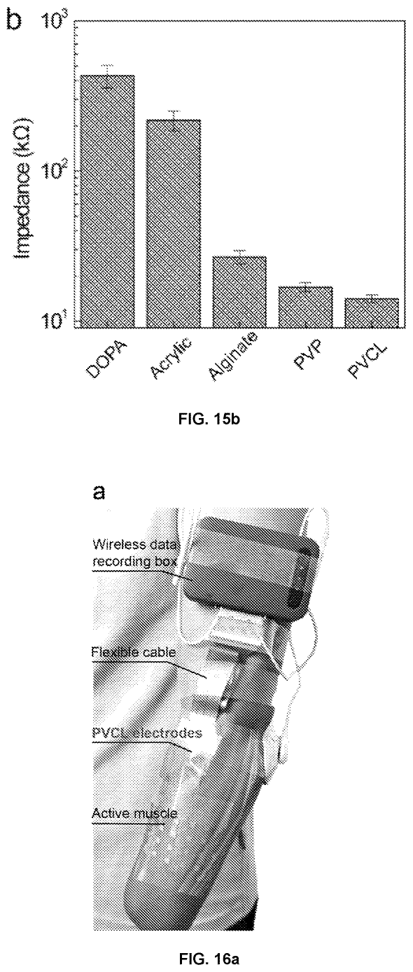

The adhesive force between electrode and skin is the macroscopical evidence for the molecule anchoring (FIG. 2(b)) due to the strong interaction between the gel and SC matrix. Consistent with the simulation results in FIG. 6f standard 90° peel-off test on biomimetic skin made from fibroin showed PVCL was most adhesive (~189 N/m; FIG. 12b and FIG. 13). Similarly, in the 180° peel-off test, the PVCL gel remained superior to others (FIG. 14). While all five electrodes had an intrinsic impedance of 200-300) at 1 Hz (FIG. 15a), PVCL electrodes displayed the lowest impedance on normal (~14 kΩ) and amputee (~20 kΩ) skin at 1 Hz (FIG. 12c, FIG. 1c and FIG. 15b). These values, which are much closer to the intrinsic impedance of human skin (6-10 kΩ) than those achieved by the previous electrodes, allow low-level (≥1.5% MVC) myoelectric signals that approach human limits (~1% MVC) to be detected (FIG. 12d-e). The critical reason for that is the high ionic-electronic coupling efficiency of the formed EDLs through the molecule anchoring process. The impedance of raw gels without NaCl is 10⁷Ω at 1 Hz. Together, these results show that anchoring PVCL to the SC improves ionic-electronic coupling and significantly lowers the impedance on skin.

Severe signal loss due to the high bioelectric impedance always happened when the other four types of electrodes monitored the ultralow-level muscle contracted (below 10% MVC). As an essential parameter to describe the fidelity of the electrodes, we measured the signal-to-noise ratio for recorded myoelectric signals below 30% MVC (FIG. 12(f)). Except for PVCL electrode, whose SNR is 5.02 for 1.7% MVC signals and 3.725 for 1.5% MVC, all electrodes had a SNR of below 5 for <2% MVC signals. The traditional recognition algorithm based on raw myoelectric signals, such as Linear Discriminant Analysis (LDA), cannot be employed to ensure high recognition accuracy for the weak activities of the amputee's phantom limb. As the signal source for myoelectric control requires a SNR of above 5, PVCL electrode has the potential to detect ultralow myoelectric signals for fine prosthetic control.

### Example 6. Convolutional Neural Network (CNN) Based Image Recognition Algorithm

Convolutional neural network (CNN), was firstly proved to be effective on hand-written figure recognition by Lecun on the MNIST dataset (http://yann.lecun.com/exdb/mnist/). A convolutional network is considered to have the ability to extract spatial features of images.

### Convolution Neural Network (CNN)

To use convolution neural network (CNN) for image recognition, we converted the myoelectric signals into an image through 1×n×m pixel mapping, where n is the number of channels on the electrode array and m is the time taken to record each muscle contraction for an intended finger and wrist movement (in our work, m was 200 ms). The size of an electrode array depends on the size of the muscle being monitored (maximum data processing capability, nₘₐₓ, of our system is 128). As signal source for training, we defined myoelectric signals from low-level (≤20% MVC), high-level (≥20% MVC) and maximum level (≥100% MVC) muscle contraction.

By designing the model of Convnet, a basic and complex feature of the myoelectric mapping can be detected and learned with the convolutional operation. The basic Convnet includes several layers: conv2D layers, maxpooling layers, dense layers, dropout layers, batch normalization layers and softmax layers.

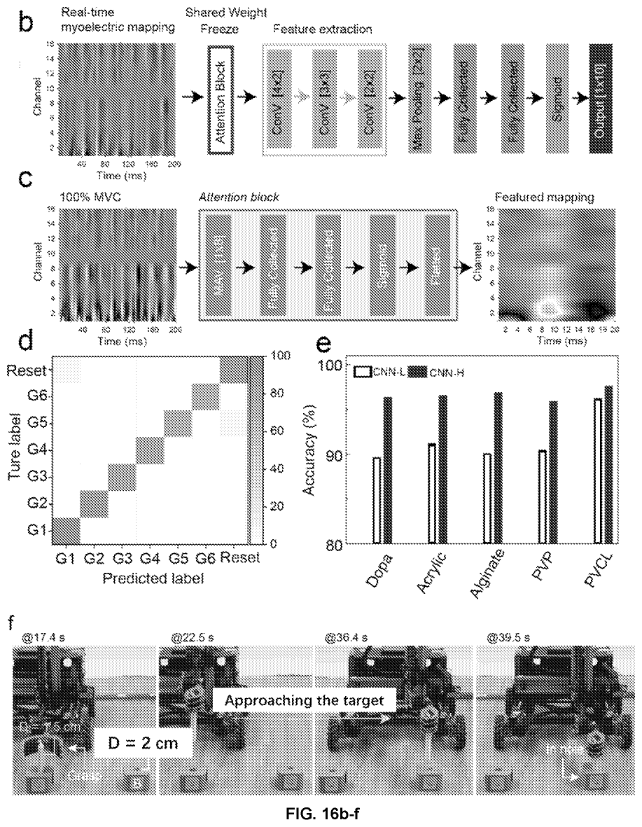

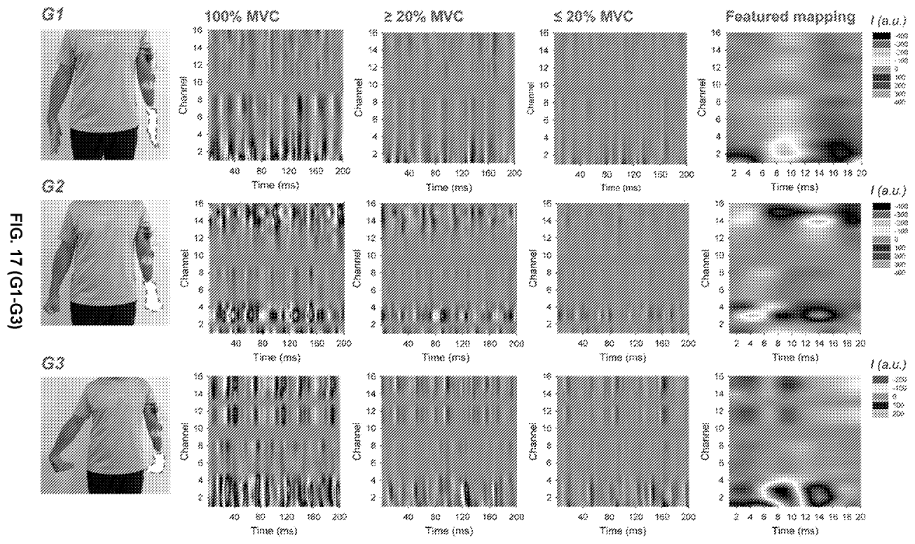

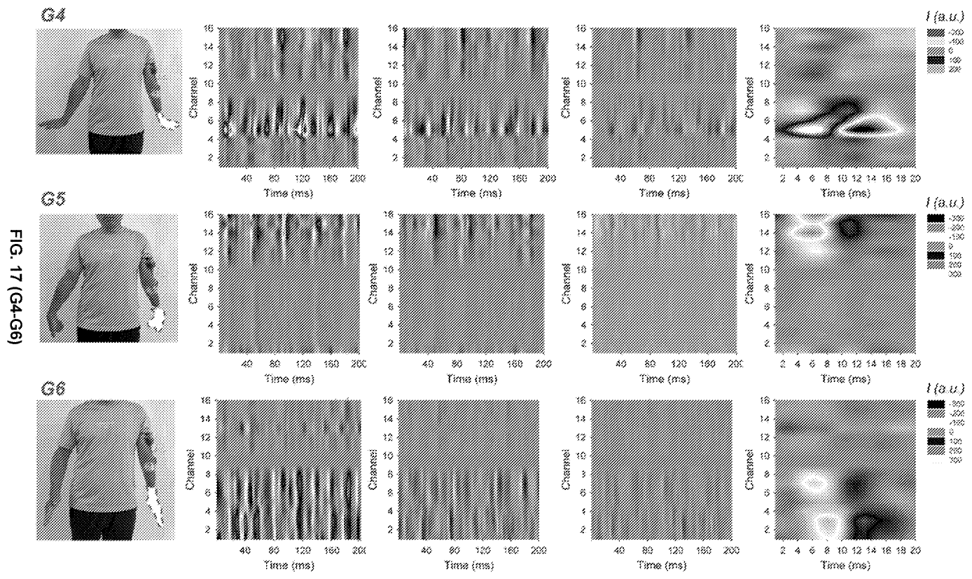

ReLU functions were adopted which included the nonlinear opponent to the network. Adaptive moment estimation (Adam) was selected as the network optimizer. The structure of the Convnet is shown in FIG. 16b. For the conv3d layers, the first conv2D layer's filter number was set to 16, the size of the kernel was set to 4×2; the second conv2D layer's filter number was set to 32, the size of the kernel was 3×3, and the size of the third kernel was 2×2. The first dense layer was set to have 64 units and the second was with 10 units, which is equal to the number of classified movements.

### Results and Discussion

Our CNN-based myoelectric mapping recognition included several layers that extracted features from the myoelectric map (conv2D layers), reduced the parameters of the model (maxpooling layer), sped up the network convergence (batch normalization layer), prevented overfitting (dropout layers) and output the predicted tensor (dense and softmax layers) (FIG. 16b). The conv2d layers were mainly used to do the convolutional operation which extracted the features from the myoelectric mapping. The maxpooling layers did the operation of down sampling and reduced the parameters of the model. Batch normalization layers prevented gradient from vanishing and made the network converging faster. Dropout layers randomly deactivated neural cells which solved the problems of overfitting. Through dense and softmax layers, the Convnet finally output the predicted tensor with the number equal to the number of the classes.

### Example 7. Introduction of Attention Mechanism on the Myoelectric Mapping

To improve the processing efficiency and classification accuracy of ultra-low myoelectric signals with low (<5) SNR, we designed an attention block (MAV block) that contributed to the network in two ways: (1) it evaluated the importance of each channel and enhanced the channel with apparent features while suppressing the channel without apparent features; and (2) it extracted MAV features from different channels of myoelectric signals and preserved the features for transfer learning so that new tasks could be performed accurately using a shorter training period (FIG. 16c). As the same muscle was used in different finger and wrist movements, the attention block evaluated and weighed each muscle channel by measuring the mean average value (MAV). The block enhanced the weight of important channels and reduced the unimportant ones.

### Attention Block

In the general task of attention block, people usually focus on the different and similar features between the source and target domains. A similar feature should remain, and the difference should be learned and calibrated. Even the small changes in muscle contraction intensity of tasks will hugely impact gesture classification. Due to the independence of the myoelectric gesture samples from various contraction intensities, it's hard to let the CNN network learn the obvious different reason for the offset, so we need to let the network focus more on the similar feature between the samples with different movement force and using fine-tune to calibrate the network.

Assumes that the myoelectric signal is n channel with m sample points in each channel. It can be seen as an input image (Eq. (3)).

$$I ∈ {R}^{1 \times m \times n} \qquad ( 3 )$$

For the channel, the mean average value (MAV) was firstly calculated (Eq. (4)).

$${mav}_{p} = mean {\sum }_{i = 1}^{m} \vert {X}_{i} \vert \qquad ( 4 )$$

Then, the raw MAV value was obtained by Eq. (5).

$${F}_{r} = [ m a {v}_{p} , m a {v}_{p} , … , {mav}_{p} ] \qquad ( 5 )$$

Through 2 MLP layers and activated by sigmoid function, eight weighted vector was obtained by Eq. (6),

$${F}_{r} = σ ( MLP ( F ) ) ∈ {R}^{1 \times n} \qquad ( 6 )$$

where σ stands for the sigmoid function.

Finally the output image was expressed by Eq. (7),

$${I}_{aug} = {F}_{scale} ( {F}_{r} , I ) \qquad ( 7 )$$

Where F_{scale} stands for the column-wised multiplication.

### Results and Discussions

The attention mechanism has been widely used in deep learning to improve the performance of the network and achieve higher classification accuracy. We designed an attention block, named MAV block. The structure of the MAV block is shown in FIG. 16c.

After extracting the same features from 100% MVC myoelectric maps, we obtained a series of activated electrodes with the related weight for every finger and wrist movement. The 100% MVC maps served to exclude the influence of signals with low SNR. Using the output after MAV block extraction (called featured mapping) as an initial judgement for real-time myoelectric mapping can theoretically decrease the delay time during recognition from 200 ms to 20 ms.

### Example 8. PVCL-Based Gel Electrodes Array for Measurement of Low-Level Myoelectric Signals

As proof of concept, we built arrays of gel electrodes based on PVCL and used them to measure low-level myoelectric signals from the stump of an amputee. The electrodes were connected to a wireless data recording box via a flexible cable and the measured myoelectric signals were used to control a prosthetic arm, which in our work was represented by a robotic car (FIG. 16). Here, we focus on using the low-level myoelectric signals to enable dexterous finger movements such as grasping with the prosthetic.

### Fabrication of Gel Electrodes

Gel electrodes were fabricated by following the protocols in Examples 1 and 5.

### Image Recognition by CNN

CNN-based myoelectric mapping recognition was carried out by following the protocol in Example 6. The processing efficiency and classification accuracy of ultra-low myoelectric signals with low (<5) SNR was improved by subjecting the myoelectric pixel image to the MAV attention block by following the protocol in Example 7.

### Myoelectric Control of a Below-Elbow Amputee Driving the Arm of a Robotic Car

We applied the system to enable a below-elbow amputee to grasp and place a candle into a hole. Using prosthetic fingers to grasp small objects with reasonable force and approach a target with high precision represents a common everyday activity for an amputee.

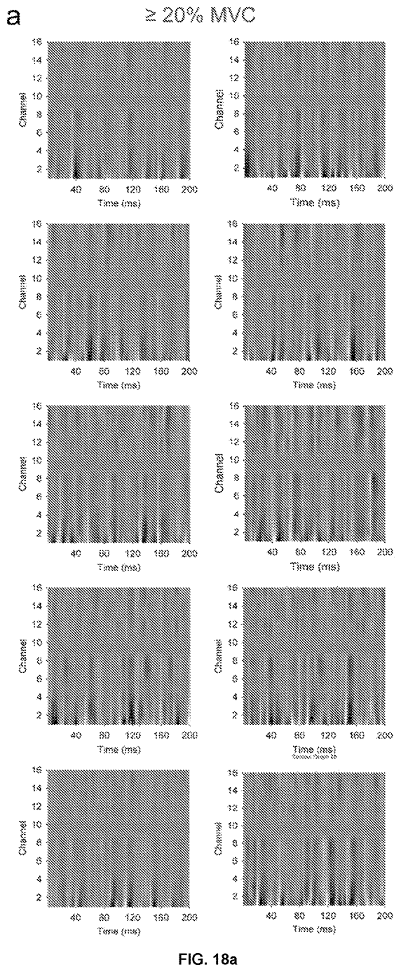

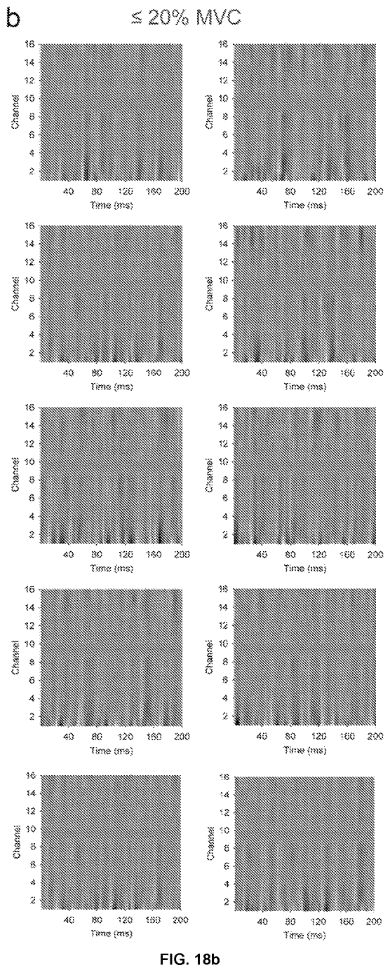

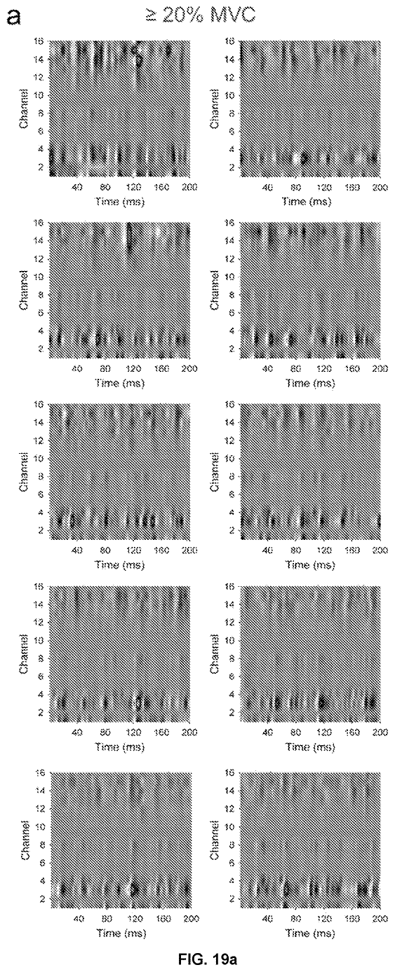

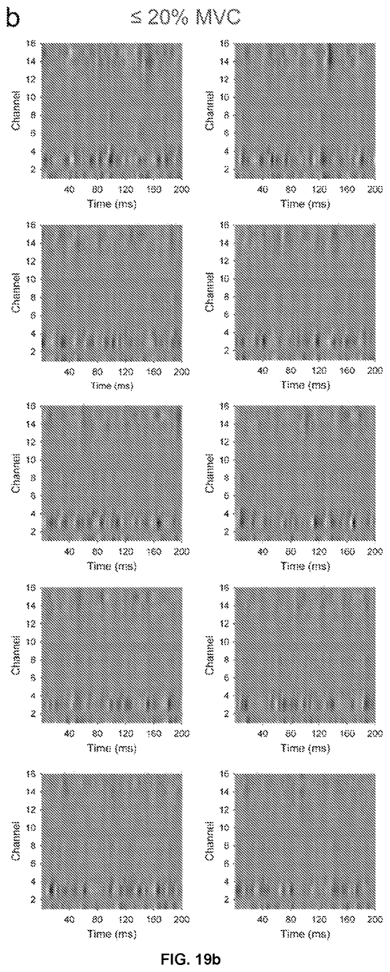

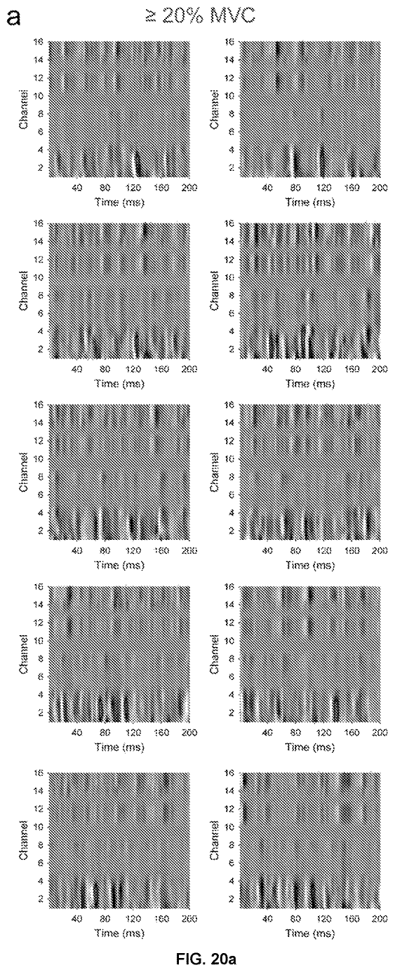

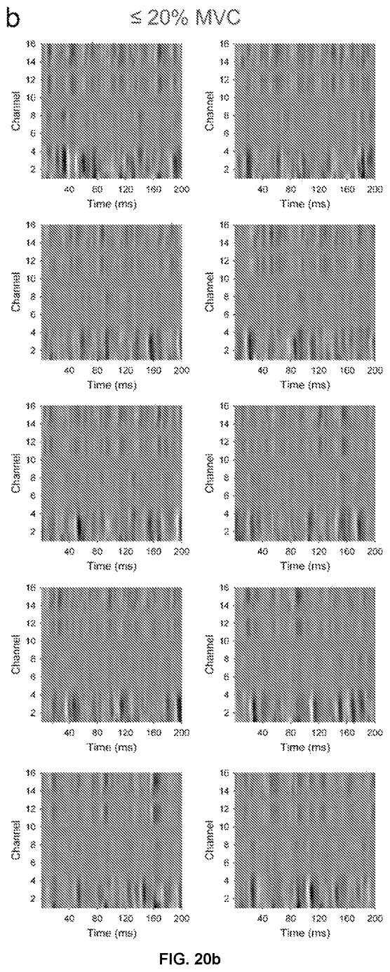

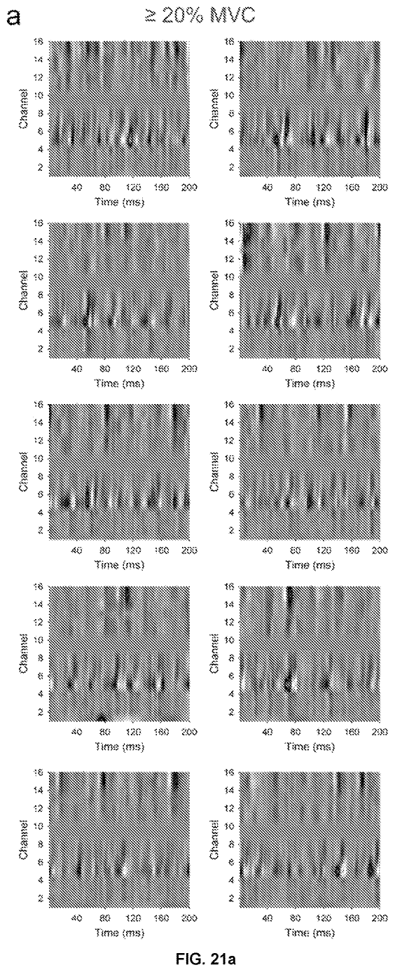

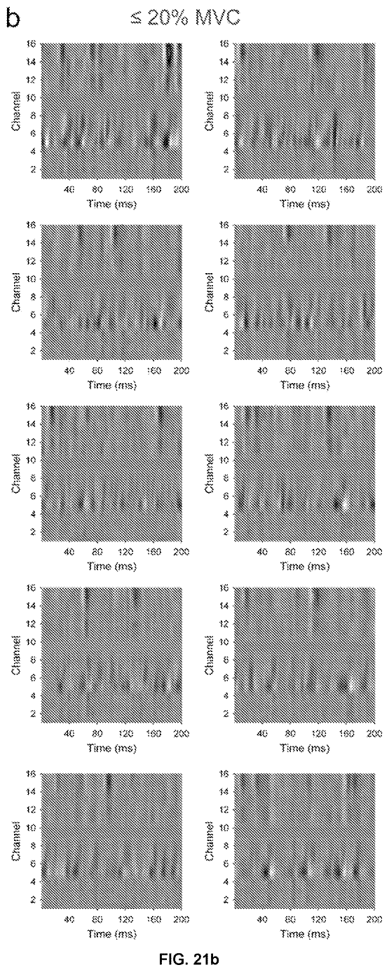

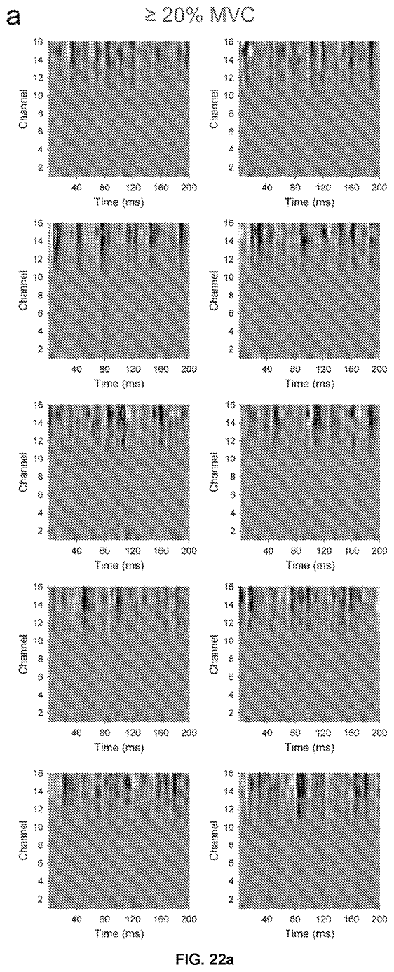

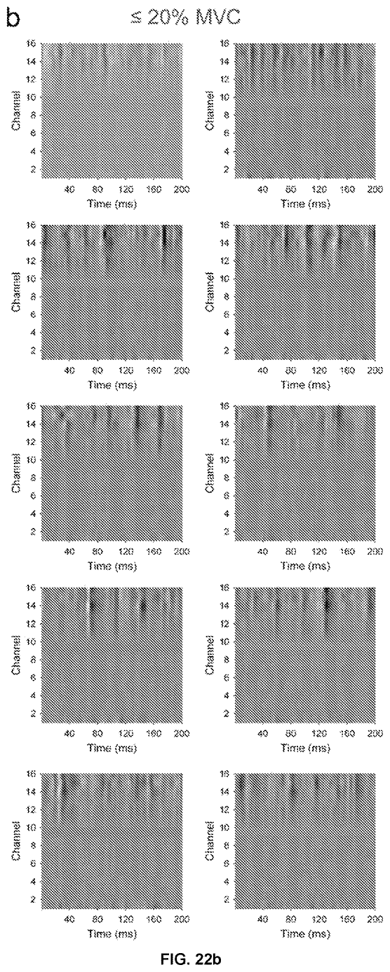

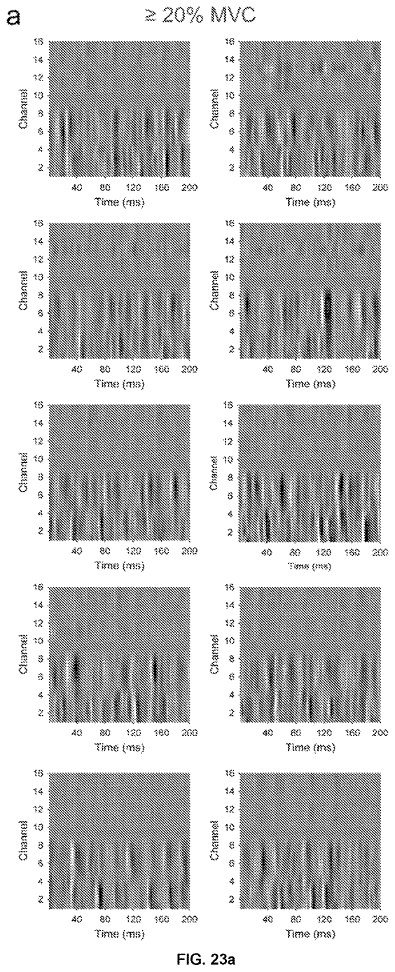

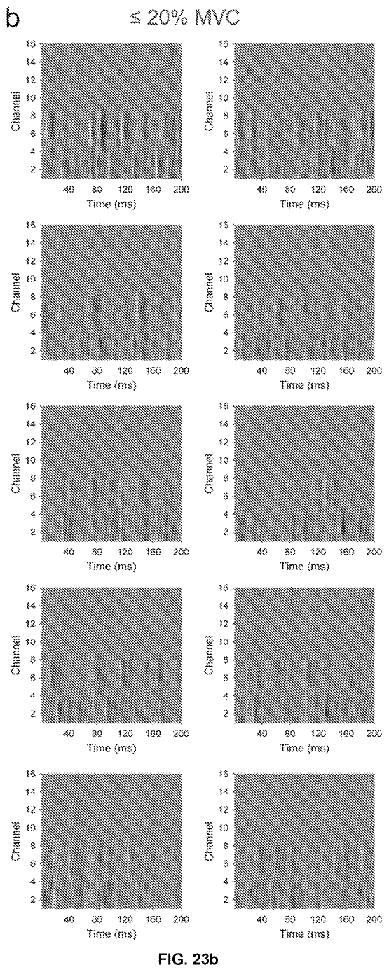

To demonstrate precision in the prosthetic control, the diameter of the hole (2 cm) is made only slightly larger than that of the candle (1.5 cm). Six finger and wrist movements (G1-G6) initiated by the amputated limb (FIG. 17) were used to drive the arm of the robotic car to release (G1, FIG. 18), grasp (G2, FIG. 19), move left (G3, FIG. 20), move right (G4, FIG. 21), move upwards (G5, FIG. 22) and downwards (G6, FIG. 23).

### Results and Discussion

Of all 5 electrodes, PVCL electrode displayed the highest CNN recognition accuracy (97.6%) for low-level signals (FIG. 16d-e and Tables 1 and 2), allowing the robotic arm to successfully grasp and place the candle into the hole (FIG. 16f). Between 22.5 s to 36.4 s, the robotic car used ultralow-level and high-quality myoelectric mappings to dexterously approach the small hole. For the robotic arm to grasp the candle, the strength of the muscle contractions for the intended movements generated by the amputated limbs must match the strength of normal finger movements. No grasping will occur if there is a mismatch. Together, our results demonstrated that PVCL electrodes anchored firmly to the skin can detect low-level myoelectric signals that are suitable for CNN-based recognition and fine prosthesis control.

TABLE 1
Confusion Matrix of high-level muscle
contraction (≤20% MVC) using CNN

| | G1 | G2 | G3 | G4 | G5 | G6 | Reset |
|---|---|---|---|---|---|---|---|
| G1 | 107 | 0 | 0 | 0 | 0 | 0 | 4 |
| G2 | 0 | 96 | 0 | 0 | 0 | 0 | 0 |
| G3 | 0 | 0 | 87 | 0 | 0 | 0 | 0 |
| G4 | 0 | 0 | 0 | 88 | 0 | 0 | 3 |
| G5 | 0 | 0 | 0 | 0 | 75 | 0 | 0 |
| G6 | 0 | 0 | 0 | 0 | 3 | 73 | 0 |
| Reset | 0 | 2 | 2 | 2 | 4 | 1 | 95 |

TABLE 2
Confusion Matrix of high-level muscle
contraction (≥20% MVC) using CNN

| | G1 | G2 | G3 | G4 | G5 | G6 | Reset |
|---|---|---|---|---|---|---|---|
| G1 | 107 | 0 | 0 | 0 | 0 | 0 | 4 |
| G2 | 0 | 96 | 0 | 0 | 0 | 0 | 0 |
| G3 | 0 | 0 | 87 | 0 | 0 | 0 | 0 |
| G4 | 0 | 0 | 0 | 88 | 0 | 0 | 3 |
| G5 | 0 | 0 | 0 | 0 | 65 | 0 | 0 |
| G6 | 0 | 0 | 0 | 0 | 3 | 73 | 0 |
| Reset | 0 | 2 | 2 | 2 | 4 | 3 | 95 |

In summary, we present a strategy to improve the detection and fidelity of low-level myoelectric signals by developing PVCL gel electrodes that anchor to the SC. Such firm anchoring enhances ionic-electronic coupling and minimizes the impedance on amputated skin (down to 20 kΩ). Our electrode, whose overall mechanical and electrical performance exceeds those of other gel electrodes (Table 3), can detect ultralow (1.5% MVC) myoelectric signals that approach human limits. We designed a CNN-based mapping recognition algorithm that can classify and predict with 97.6% accuracy the intended finger and wrist movements from myoelectric signals obtained using our PVCL electrodes. With such high-fidelity signals and classification accuracy, our system enabled an amputee to drive a prosthesis with finger dexterity. Such fine movements are expected to improve the quality of life of amputees who depend on prostheses to accomplish day-to-day tasks.

TABLE 3
Mechanical and electrical performance of five gel electrodes.

| | Alg-PAAm | | DOPA | | Acrylic | | PVP | | PVCL | |
|---|---|---|---|---|---|---|---|---|---|---|
| LogP | −3.84 | | −2.2 | | 0.44 | | 0.74 | | 1.75 | |
| ΔG | 40.64 | k_{B}T | 12.36 | k_{B}T | 9.81 | k_{B}T | 10.05 | k_{B}T | 6.57 | k_{B}T |
| Anchoring depth in | 0 | μm | 0.97 | μm | 1.18 | μm | 2.43 | μm | 3.07 | μm |
| biomimetic skin | | | | | | | | | | |
| Bioelectronic | 26 | kΩ | 431 | kΩ | 217 | kΩ | 16 | kΩ | 14 | kΩ |
| impedance on | | | | | | | | | | |
| normal skin | | | | | | | | | | |
| Adhesive force | 90 | N/m | 30 | N/m | 45 | N/m | 151 | N/m | 190 | N/m |

| | | | | | |
|---|---|---|---|---|---|
| Detecting | 2.1% | / | 10.3% | 1.7% | 1.5% |
| limitation | | | | | |
| Accuracy rate | 89.61% | 91.2% | 90.05% | 90.47% | 97.59% |
| based on CNN | | | | | |

1. An ionic conducting gel that comprises:
a polymeric adhesive material in an amount of from 5 to 20 wt % of the gel;
a metal salt in the amount of from 2 to 10 wt % of the gel; and
the balance being a hydrophilic polymeric material, wherein a gel matrix is formed by the polymeric adhesive material and the hydrophilic polymeric material, and the metal salt is distributed throughout the gel matrix.

2. The ionic conducting gel according to claim 1, wherein the number average molecular weight of the hydrophilic polymeric material is from 200 to 15,000 Daltons.

3. The ionic conducting gel according to claim 1, wherein the hydrophilic polymeric material is a polyethylene glycol or a polyalcohol.

4. The ionic conducting gel according to claim 3, wherein the hydrophilic polymeric material is a polyethylene glycol.

5. The ionic conducting gel according to claim 1, wherein the polymeric adhesive material is selected from one or more of the group consisting of a polysaccharide, a cellulose hydrogel, a protein hydrogel, a nucleic acid gel, a biohydrogel and, more particularly, poly(N-vinyl caprolactam) (PVCL), polyvinyl pyrrolidone (PVP), polydopamine, and a polymeric acrylic.

6. The ionic conducting gel according to claim 5, wherein the polymeric adhesive material is selected from one or more of the group consisting of poly(N-vinyl caprolactam) (PVCL), polyvinyl pyrrolidone (PVP), polydopamine, and a polymeric acrylic.

7. The ionic conducting gel according to claim 6, wherein the polymeric adhesive material is poly(N-vinyl caprolactam) (PVCL).

8. The ionic conducting gel according to claim 1, wherein the polymeric adhesive material represents about 10 wt % of the total weight of the gel.

9. The ionic conducting gel according to claim 1, wherein the metal salt is selected from one or more of the group consisting of a an iron salt, a sodium salt, a potassium salt, a calcium salt, and a magnesium salt.

10. The ionic conducting gel according to claim 9, wherein metal salt is sodium chloride.

11. The ionic conducting gel according to claim 1, wherein the metal salt is represents about 5 wt % of the total weight of the gel.

12. A flexible electrode device, comprising:
a flexible substrate material having a first surface and a second surface;
a metal electrode pad on the first surface of the substrate; and
an ionic conducting gel layer that covers the metal electrode pad, wherein the ionic conducting gel is as described in claim 1.

13. The electrode device according to claim 12, wherein the device further comprises two or more metal electrode pads on the first surface of the substrate,
where each of the two or more metal electrode pads is electrically connected to at least one conductive pathway,
where each of the two or more metal electrode pads is covered by an ionic conducting gel layer, and
where each conductive pathway is covered by an insulating material.

14. The electrode device according to claim 12, wherein the impedance of the electrode device is approximately from 10⁻³ to 10⁵Ω at from 1 to 10⁶ Hz.

15. The electrode device according to claim 13, wherein the two or more metal electrode pads is from 2 to 512.

16. A prosthetic control system comprising one or more electrode devices according to claim 12.

17. The ionic conducting gel according to claim 4, wherein the polyethylene glycol has a number average molecular weight of 200.

18. The electrode device according to claim 13, wherein the two or more metal electrode pads is from 8 to 256 metal electrode pads.

19. The electrode device according to claim 13, wherein the two or more metal electrode pads is from 15 to 128 metal electrode pads.
